(19) United States
(12) Patent Application Publication
(10) Pub. No.: US 2026/0015344 A1
(43) Pub. Date: Jan. 15, 2026

(54) PAN-KRAS INHIBITORS

(71) Applicant: Mirati Therapeutics, Inc., Princeton, NJ (US)

(72) Inventors: Svitlana KULYK, San Diego, CA (US); Xiaolun WANG, San Diego, CA (US)

(21) Appl. No.: 18/875,573

(22) PCT Filed: Jun. 13, 2023
(86) PCT No.: PCT/US2023/025191

§ 371 Date: Dec. 16, 2024

Related U.S. Application Data
(60) Provisional application No. 63/352,490, filed on Jun. 15, 2022.

(51) Int. Cl. C07D 413/14; A61K 31/506; A61K 31/551; A61K 31/553; C07D 417/14; C07D 471/08; C07D 471/10; C07D 487/04; C07D 487/08; C07D 513/10

(52) U.S. Cl. CPC C07D 413/14; A61K 31/506; A61K 31/551; A61K 31/553; C07D 417/14; C07D 471/08; C07D 471/10; C07D 487/04; C07D 487/08; C07D 513/10

(57) ABSTRACT

The present invention relates to compounds that inhibit at least one of KRas G12A, KRas G12C, KRas G12D, KRas G12R, KRas G12S, KRas G12V, KRas G13D and KRas Q61H, pharmaceutical compositions comprising the compounds and methods of use therefor.

### FIELD OF THE INVENTION

The present invention relates to compounds that inhibit multiple mutated forms of KRas, i.e., pan-KRas inhibitors. In particular, the present invention relates to pan-KRas compounds, pharmaceutical compositions comprising the compounds and methods of use therefor.

### BACKGROUND OF THE INVENTION

Kirsten Rat Sarcoma 2 Viral Oncogene Homolog (“KRas”) is a small GTPase and a member of the Ras family of oncogenes. KRas serves as a molecular switch cycling between inactive (GDP-bound) and active (GTP-bound) states to transduce upstream cellular signals received from multiple tyrosine kinases to downstream effectors to regulate a wide variety of processes, including cellular proliferation (e.g., see Alamgeer et al., (2013) Current Opin Pharmcol. 13:394-401).

The role of activated KRas in malignancy was observed over thirty years ago (e.g., see Santos et al., (1984) Science 223:661-664). Aberrant expression of KRas accounts for up to 20% of all cancers and oncogenic KRas mutations that stabilize GTP binding and lead to constitutive activation of KRas. KRas mutations at codons 12, 13, 61 and other positions of the KRas primary amino acid sequence are present in 88% of all pancreatic adenocarcinoma patients, 50% of all colon/rectal adenocarcinoma patients, and 32% lung adenocarcinoma patients (e.g., see Prior et all., (2020) Cancer Res 80:2969-74). A recent publication also suggested wild type Kras inhibition could be a viable therapeutic strategy to treat KRas^{WT} dependent cancers (e.g., see Bery et al., (2020) Nat. Commun. 11:3233).

The well-known role of KRas in malignancy and the discovery of these frequent mutations in KRas in various tumor types made KRas a highly attractive target of the pharmaceutical industry for cancer therapy. Notwithstanding thirty years of large-scale discovery efforts to develop inhibitors of KRas for treating cancer, no KRas inhibitor has yet demonstrated sufficient safety and/or efficacy to obtain regulatory approval (e.g., see McCormick (2015) Clin Cancer Res. 21 (8):1797-1801).

Compounds that inhibit KRas activity are still highly desirable and under investigation, including those that disrupt effectors such as guanine nucleotide exchange factors (e.g., see Sun et al., (2012) Agnew Chem Int Ed Engl. 51(25):6140-6143 doi: 10.1002/anie201201358) as well recent advances in the covalent targeting of an allosteric pocket of KRas G12C (e.g., see Ostrem et al., (2013) Nature 503:548-551 and Fell et al., (2018) ACS Med. Chem. Lett. 9:1230-1234). Clearly there remains a continued interest and effort to develop inhibitors of KRas, particularly inhibitors of activating KRas mutants.

Thus, there is a need to develop new pan-KRas inhibitors that demonstrate sufficient efficacy for treating KRas-mediated cancers.

### SUMMARY OF THE INVENTION

In one aspect of the invention, compounds are provided that inhibit KRas activity. In certain embodiments, the compounds are represented by Formula (I):

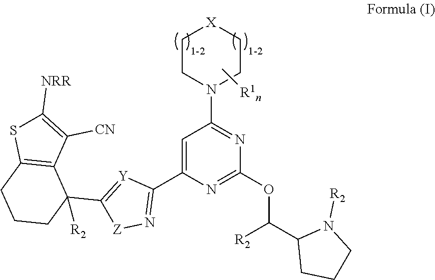

or a pharmaceutically acceptable salt thereof, wherein:
X is CR, O or N;
Y is CR or N;
Z is O or S;
n is an integer from 1 to 4;
each R is independently H or C1-C3 alkyl;
R¹ is C1-C3 alkyl or hydroxy; or
n is at least two, and two R¹s optionally join to form a methylene or ethylene bridge; or
n is at least two, and two R¹s optionally join to form a spiro or fused ring, where the ring is heterocyclic or heteroaryl, and where the ring is optionally substituted with 1-2 substituents selected from oxo and —C(O)N(CH3)(CH3);
each R² is independently C1-C3 alkyl.

In another aspect of the invention, pharmaceutical compositions are provided comprising a therapeutically effective amount of a compound of the present invention or a pharmaceutically acceptable salt thereof and a pharmaceutically acceptable excipient.

In yet another aspect of the invention, methods for inhibiting the activity of cells containing wild type KRas or one or more KRas mutations, for instance the KRas mutations G12A, G12C, G12D, G12R, G12S, G12V, G13D and/or Q61H, in a in a cell, comprising contacting the cell with a compound of Formula (I), or a pharmaceutically acceptable salt thereof, or a pharmaceutical composition thereof as defined herein. In one embodiment, the contacting is in vitro. In one embodiment, the contacting is in vivo.

Also provided herein is a method of inhibiting cell proliferation, in vitro or in vivo, the method comprising contacting a cell with an effective amount of a compound of Formula (I), or a pharmaceutically acceptable salt thereof, or a pharmaceutical composition thereof as defined herein.

Also provided are methods for treating cancer in a patient comprising administering a therapeutically effective amount of a compound or pharmaceutical composition of the present invention or a pharmaceutically acceptable salt thereof to a patient in need thereof.

Also provided herein is a method of treating a KRas wild type, KRas G12A, G12C, G12D, G12R, G12S, G12V, G13D and/or Q61H-associated disease or disorder in a patient in need of such treatment, the method comprising administering to the patient a therapeutically effective amount of a compound of Formula (I), or a pharmaceutically acceptable salt thereof, or a pharmaceutical composition thereof as defined herein.

Also provided herein is a compound of Formula (I), or a pharmaceutically acceptable salt thereof, or a pharmaceutical composition thereof as defined herein for use in therapy.

Also provided herein is a compound of Formula (I), or a pharmaceutically acceptable salt thereof or a pharmaceutical composition thereof as defined herein for use in the treatment of cancer.

Also provided herein is a compound of Formula (I), or a pharmaceutically acceptable salt thereof for use in the inhibition of KRas wild type or multiple types of KRas mutations, for instance KRas G12A, G12C, G12D, G12R, G12S, G12V, G13D and/or Q61H mutations.

Also provided herein is a compound of Formula (I), or a pharmaceutically acceptable salt thereof or a pharmaceutical composition thereof as defined herein, for use in the treatment of a KRas wild type associated disease or disorder or a KRas mutation G12A, G12C, G12D, G12R, G12S, G12V, G13D and/or Q61H-associated disease or disorder.

Also provided herein is the use of a compound of Formula (I), or a pharmaceutically acceptable salt thereof, as defined herein in the manufacture of a medicament for the treatment of cancer.

Also provided herein is a use of a compound of Formula (I), or a pharmaceutically acceptable salt thereof, as defined herein in the manufacture of a medicament for the inhibition of activity of the wild type form of KRas or mutated forms of KRas, including the mutations: G12A, G12C, G12D, G12R, G12S, G12V, G13D and/or Q61H.

Also provided herein is the use of a compound of Formula (I), or a pharmaceutically acceptable salt thereof, as defined herein, in the manufacture of a medicament for the treatment of a KRas wild type associated disease or disorder or a KRas G12A, G12C, G12D, G12R, G12S, G12V, G13D and/or Q61H-associated disease or disorder.

Also provided herein is a method for treating cancer in a patient in need thereof, the method comprising (a) determining that the cancer is associated with KRas wild type or a KRas G12A, G12C, G12D, G12R, G12S, G12V, G13D and/or Q61H mutation (i.e., a KRas G12A, G12C, G12D, G12R, G12S, G12V, G13D and/or Q61H-associated cancer); and (b) administering to the patient a therapeutically effective amount of a compound of Formula (I), or a pharmaceutically acceptable salt thereof, or a pharmaceutical composition thereof.

One potential utility of the herein-described pan-KRas inhibitors is for the treatment of cancers that develop resistance following long-term treatment with KRas G12C inhibitors. Thus, embodiments of the invention include those wherein a patient suffering from cancer is treated with a herein-described pan-KRas inhibitor after treatment with a G12C inhibitor becomes ineffective or less effective due to the emergence of resistance-imparting mutations.

Treatment of KRas G12C mutant cancers with covalent KRas G12C inhibitors such as adagrasib (MRTX849) or sotorasib (AMG510) may result in the incorporation of additional mutations that confer resistance to adagrasib. These mutations could confer resistance through numerous mechanisms.

Mutations that change the mutant cysteine at codon 12 to another amino acid would render the current covalent KRas G12C inhibitors ineffective since current inhibitors make a covalent bond with the mutant cysteine amino acid side chain. Likewise, in patients that have one wild type KRas allele in addition to the KRas G12C-mutant allele, mutations in the wild type codon 12 glycine to another codon would allow bypass signaling in these tumors through the novel mutant protein. The repertoire of codon 12 mutations that can occur with a single nucleotide substitution in the wild type gene (glycine codon) includes mutations commonly observed in in cancer such as G12S, G12V, G12R, G12C. The repertoire of codon 12 mutations that can occur with single nucleotide base substitutions of the cysteine codon 12 include mutations not frequently observed in cancer, G12Y, G12F and G12W, in addition to G12S and G12R.

Second-site mutations may also occur in another location in the KRas G12C mutant gene that confers resistance to KRas G12C inhibitor treatment. These mutations may confer resistance through different mechanisms. RAS proteins are small GTPases that normally cycle between an active, GTP-bound state and an inactive, GDP-bound state. RAS proteins are loaded with GTP through guanine nucleotide exchange factors (GEFs; e.g., SOS1) which are activated by upstream receptor tyrosine kinases, triggering subsequent interaction with effector proteins that activate RAS-dependent signaling. RAS proteins hydrolyze GTP to GDP through their intrinsic GTPase activity which is dramatically enhanced by GTPase-activating proteins (GAPs). Mutations at codons 12 and 13 in RAS proteins impair GAP-stimulated GTP hydrolysis leaving RAS predominantly in the GTP-bound, active state. Covalent KRas G12C inhibitors in current clinical development only bind GDP-bound KRas G12C. Mutations such as Q61 codon mutations, which may or may not occur on the same allele as the G12C mutation, reduce the intrinsic GTPase activity of KRas and may represent a mechanism of resistance to KRas G12C inhibitor treatment by shifting KRas into the GTP-loaded state where it is not susceptible to covalent inhibition. Co-mutations such as R68, H95 and Y96 may be present along with the KRas G12C mutation and may diminish the binding affinity of KRas G12C inhibitors to the Switch II binding pocket.

The herein-described pan-KRas inhibitors may demonstrate activity against common as well as uncommon codon 12 mutations or mutations that occur in the KRas protein that diminish binding of KRas G12C inhibitors to the KRas protein.

Also provided herein is a process for preparing a compound of Formula (I), or a pharmaceutically acceptable salt thereof.

Also provided herein is a compound of Formula (I), or a pharmaceutically acceptable salt thereof obtained by a process of preparing the compound as defined herein.

### DETAILED DESCRIPTION OF THE INVENTION

The present invention relates to inhibitors of KRas wild type and/or multiple mutated forms of KRas, for instance KRas G12A, G12C, G12D, G12R, G12S, G12V, G13D and/or Q61H mutations. In particular, the present invention relates to compounds that inhibit the activity of KRas wild type and/or KRas mutations such as G12A, G12C, G12D, G12R, G12S, G12V, G13D and/or Q61H, pharmaceutical compositions comprising a therapeutically effective amount of the compounds and methods of use therefor.

### Definitions

Unless defined otherwise, all technical and scientific terms used herein have the same meaning as is commonly understood by one of skill in the art to which this invention belongs. All patents, patent applications, and publications referred to herein are incorporated by reference.

As used herein, “wild type KRas” refers to a non-mutant form of a mammalian KRas protein. The assignment of amino acid codon and residue positions for human KRas is based on the amino acid sequence identified by UniProtKB/Swiss-Prot P01116: Variantp.Glyl2Asp. As used herein, a “wild type KRas inhibitor” refers to compounds of the present invention that are represented by Formula (I), as described herein. These compounds are capable of negatively modulating or inhibiting all or a portion of the enzymatic activity of wild type KRas G12A. A “wild type KRas-associated disease or disorder” as used herein refers to diseases or disorders associated with or mediated by or having wild type KRas. A non-limiting example of a wild type KRas-associated disease or disorder is a wild type KRas-associated cancer.

As used herein, “KRas G12A” refers to a mutant form of a mammalian KRas protein that contains an amino acid substitution of an alanine for a glycine at amino acid position 12. The assignment of amino acid codon and residue positions for human KRas is based on the amino acid sequence identified by UniProtKB/Swiss-Prot P01116: Variantp.Glyl2Asp. As used herein, a “KRas G12A inhibitor” refers to compounds of the present invention that are represented by Formula (I), as described herein. These compounds are capable of negatively modulating or inhibiting all or a portion of the enzymatic activity of KRas G12A. A “KRas G12A-associated disease or disorder” as used herein refers to diseases or disorders associated with or mediated by or having a KRas G12A mutation. A non-limiting example of a KRas G12A-associated disease or disorder is a KRas G12A-associated cancer.

As used herein, “KRas G12C” refers to a mutant form of a mammalian KRas protein that contains an amino acid substitution of a cysteine for a glycine at amino acid position 12. The assignment of amino acid codon and residue positions for human KRas is based on the amino acid sequence identified by UniProtKB/Swiss-Prot P01116: Variantp.Glyl2Asp. As used herein, a “KRas G12C inhibitor” refers to compounds of the present invention that are represented by Formula (I), as described herein. These compounds are capable of negatively modulating or inhibiting all or a portion of the enzymatic activity of KRas G12C. A “KRas G12C-associated disease or disorder” as used herein refers to diseases or disorders associated with or mediated by or having a KRas G12C mutation. A non-limiting example of a KRas G12C-associated disease or disorder is a KRas G12CD-associated cancer.

As used herein, “KRas G12D” refers to a mutant form of a mammalian KRas protein that contains an amino acid substitution of an aspartic acid for a glycine at amino acid position 12. The assignment of amino acid codon and residue positions for human KRas is based on the amino acid sequence identified by UniProtKB/Swiss-Prot P01116: Variantp.Glyl2Asp. As used herein, a “KRas G12D inhibitor” refers to compounds of the present invention that are represented by Formula (I), as described herein. These compounds are capable of negatively modulating or inhibiting all or a portion of the enzymatic activity of KRas G12D. A “KRas G12D-associated disease or disorder” as used herein refers to diseases or disorders associated with or mediated by or having a KRas G12D mutation. A non-limiting example of a KRas G12D-associated disease or disorder is a KRas G12D-associated cancer.

As used herein, “KRas G12R” refers to a mutant form of a mammalian KRas protein that contains an amino acid substitution of an arginine for a glycine at amino acid position 12. The assignment of amino acid codon and residue positions for human KRas is based on the amino acid sequence identified by UniProtKB/Swiss-Prot P01116: Variantp.Glyl2Asp. As used herein, a “KRas G12R inhibitor” refers to compounds of the present invention that are represented by Formula (I), as described herein. These compounds are capable of negatively modulating or inhibiting all or a portion of the enzymatic activity of KRas G12R. A “KRas G12R-associated disease or disorder” as used herein refers to diseases or disorders associated with or mediated by or having a KRas G12R mutation. A non-limiting example of a KRas G12R-associated disease or disorder is a KRas G12R-associated cancer.

As used herein, “KRas G12S” refers to a mutant form of a mammalian KRas protein that contains an amino acid substitution of a serine for a glycine at amino acid position 12. The assignment of amino acid codon and residue positions for human KRas is based on the amino acid sequence identified by UniProtKB/Swiss-Prot P01116: Variantp.Glyl2Asp. As used herein, a “KRas G12S inhibitor” refers to compounds of the present invention that are represented by Formula (I), as described herein. These compounds are capable of negatively modulating or inhibiting all or a portion of the enzymatic activity of KRas G12S. A “KRas G12S-associated disease or disorder” as used herein refers to diseases or disorders associated with or mediated by or having a KRas G12S mutation. A non-limiting example of a KRas G12S-associated disease or disorder is a KRas G12S-associated cancer.

As used herein, “KRas G12V” refers to a mutant form of a mammalian KRas protein that contains an amino acid substitution of a valine for a glycine at amino acid position 12. The assignment of amino acid codon and residue positions for human KRas is based on the amino acid sequence identified by UniProtKB/Swiss-Prot P01116: Variantp.Glyl2Asp. As used herein, a “KRas G12V inhibitor” refers to compounds of the present invention that are represented by Formula (I), as described herein. These compounds are capable of negatively modulating or inhibiting all or a portion of the enzymatic activity of KRas G12V. A “KRas G12V-associated disease or disorder” as used herein refers to diseases or disorders associated with or mediated by or having a KRas G12V mutation. A non-limiting example of a KRas G12V-associated disease or disorder is a KRas G12V-associated cancer.

As used herein, “KRas G13D” refers to a mutant form of a mammalian KRas protein that contains an amino acid substitution of an aspartic acid for a glycine at amino acid position 13. The assignment of amino acid codon and residue positions for human KRas is based on the amino acid sequence identified by UniProtKB/Swiss-Prot P01116: Variantp.Glyl2Asp. As used herein, a “KRas G13D inhibitor” refers to compounds of the present invention that are represented by Formula (I), as described herein. These compounds are capable of negatively modulating or inhibiting all or a portion of the enzymatic activity of KRas G13D. A “KRas G13D-associated disease or disorder” as used herein refers to diseases or disorders associated with or mediated by or having a KRas G13D mutation. A non-limiting example of a KRas G13D-associated disease or disorder is a KRas G13D-associated cancer.

As used herein, “KRas Q61H” refers to a mutant form of a mammalian KRas protein that contains an amino acid substitution of a histidine for a glutamine at amino acid position 61. The assignment of amino acid codon and residue positions for human KRas is based on the amino acid sequence identified by UniProtKB/Swiss-Prot P01116: Variantp.Glyl2Asp. As used herein, a “KRas Q61H inhibitor” refers to compounds of the present invention that are represented by Formula (I), as described herein. These compounds are capable of negatively modulating or inhibiting all or a portion of the enzymatic activity of KRas Q61H. A “KRas Q61H-associated disease or disorder” as used herein refers to diseases or disorders associated with or mediated by or having a KRas Q61H mutation. A non-limiting example of a KRas Q61H-associated disease or disorder is a KRas Q61H-associated cancer.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a cancer having wild type KRas or a KRas G12A, G12C, G12D, G12R, G12S, G12V, G13D and/or Q61H mutation (e.g., as determined using a regulatory agency-approved, e.g., FDA-approved, assay or kit). In some embodiments, the subject has a tumor that is positive for wild type KRas or a KRas G12A, G12C, G12D, G12R, G12S, G12V, G13D and/or Q61H mutation (e.g., as determined using a regulatory agency-approved assay or kit). The subject can be a subject with a tumor(s) that is positive for wild type KRas or a KRas G12A, G12C, G12D, G12R, G12S, G12V, G13D and/or Q61H mutation (e.g., identified as positive using a regulatory agency-approved, e.g., FDA-approved, assay or kit). The subject can be a subject whose tumors have wild type KRas or a KRas G12A, G12C, G12D, G12R, G12S, G12V, G13D and/or Q61H mutation (e.g., where the tumor is identified as such using a regulatory agency-approved, e.g., FDA-approved, kit or assay). In some embodiments, the subject is suspected of having wild type KRas or a KRas G12A, KRas G12C, KRas G12D, KRas G12R, KRas G12S, KRas G12V, KRas G13D or KRas Q61H gene-associated cancer. In some embodiments, the subject has a clinical record indicating that the subject has a tumor that has wild type KRas or a KRas G12A, G12C, G12D, G12R, G12S, G12V, G13D and/or Q61H mutation (and optionally the clinical record indicates that the subject should be treated with any of the compositions provided herein).

In some embodiments of any of the methods or uses described herein, an assay is used to determine whether the patient has wild type KRas or a KRas G12A, G12C, G12D, G12R, G12S, G12V, G13D and/or Q61H mutation using a sample (e.g., a biological sample or a biopsy sample (e.g., a paraffin-embedded biopsy sample) from a patient (e.g., a patient suspected of having wild type KRas-associated or a KRas G12A, G12C, G12D, G12R, G12S, G12V, G13D and/or Q61H-associated cancer, a patient having one or more symptoms of wild type KRas-associated or a KRas G12A, G12C, G12D, G12R, G12S, G12V, G13D and/or Q61H-associated cancer, and/or a patient that has an increased risk of developing wild type KRas-associated or a KRas G12A, G12C, G12D, G12R, G12S, G12V, G13D and/or Q61H-associated cancer) can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof.

The term “regulatory agency” is a country's agency for the approval of the medical use of pharmaceutical agents with the country. For example, a non-limiting example of a regulatory agency is the U.S. Food and Drug Administration (FDA).

The term “acyl” refers to —C(O)CH3.

The terms “C1-C6 alkyl”, “C1-C4 alkyl” and “C1-C3 alkyl” as employed herein refers to straight and branched chain aliphatic groups having from 1-6 carbon atoms, or 1-4 carbon atoms, or 1-3 carbon atoms, respectively. Examples of alkyl groups include, without limitation, methyl, ethyl, propyl, isopropyl, butyl, isobutyl, sec-butyl, tert-butyl, pentyl, and hexyl.

The terms “C1-C3 haloalkyl” and “C1-C4 haloalkyl” refer to a C1-C3 alkyl chain or C1-C4 alkyl chain, respectively, as defined herein in which one or more hydrogen has been replaced by a halogen. Examples include trifluoromethyl, difluoromethyl and fluoromethyl.

An “C1-C4 alkylene,” group is a C1-C4 alkyl group, as defined hereinabove, that is positioned between and serves to connect two other chemical groups. Exemplary alkylene groups include, without limitation, methylene, ethylene, propylene, and butylene.

The terms “C1-C3 alkoxy” and “C1-C4 alkoxy” refer to —OC1-C3 alkyl and —OC1-C4 alkyl, respectively, wherein the alkyl portion is as defined herein above.

The term “cycloalkyl” as employed herein includes saturated and partially unsaturated cyclic hydrocarbon groups having 3 to 12 carbons, for example 3 to 8 carbons, and as a further example 3 to 6 carbons, wherein the cycloalkyl group additionally is optionally substituted with one or more R⁸ or R⁹ groups as defined herein. Examples of cycloalkyl groups include, without limitation, cyclopropyl, cyclobutyl, cyclopentyl, cyclopentenyl, cyclohexyl, cyclohexenyl, cycloheptyl, and cyclooctyl. The term “cycloalkyl” also includes bridged cycloalkyls, such as bicyclo[1.1.1]pentanyl.

As used herein, the terms “C1-C3 hydroxyalkyl” and “C1-C4 hydroxyalkyl” refer to —C1-C3 alkylene-OH and —C1-C4 alkylene-OH, respectively.

As used herein, the term “C2-C4 hydroxyalkynyl” refers to —C2-C4 alkynylene-OH.

An “aryl” group is a C₆-C₁₄ aromatic moiety comprising one to three aromatic rings, which is optionally substituted with one or more R⁸ or R⁹ groups as defined herein. As one embodiment, the aryl group is a C₆-C₁₀ aryl group. Examples of aryl groups include, without limitation, phenyl, naphthyl, anthracenyl, fluorenyl, and dihydrobenzofuranyl. “Aryl” also refers to bicyclic or tricyclic ring systems in which one or two rings, respectively, of said aryl ring system may be saturated or partially saturated, and wherein if said ring system includes two saturated rings, said saturated rings may be fused or spirocyclic. An example of an aryl ring system comprising two saturated rings wherein the rings are spirocyclic includes the following ring system:

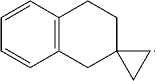 [SMILES: c1ccc2c(c1)CCC1(CC1)C2]

An “araC1-C6 alkyl” or “arylalkyl” group comprises an aryl group covalently linked to an alkyl group, either of which may independently be optionally substituted or unsubstituted. An example of an aralkyl group is (C6-C10)aryl(C1-C6)alkyl-, including, without limitation, benzyl, phenethyl, and naphthylmethyl. An example of a substituted araC1-C6 alkyl is wherein the alkyl group is substituted with hydroxyalkyl.

A “heterocyclyl” or “heterocyclic” group is a ring structure having from 3 to 12 atoms, for example 4 to 8 atoms, wherein one or more atoms are selected from the group consisting of N, O, and S wherein the ring N atom may be oxidized to N—O, and the ring S atom may be oxidized to SO or SO₂, the remainder of the ring atoms being carbon. The heterocyclyl may be a monocyclic, a bicyclic, a spirocyclic or a bridged ring system. The heterocyclic group is optionally substituted with one or more R⁸ or R⁹ groups on ring carbon or ring nitrogen at one or more positions, wherein R⁶ is as defined for Formula I. The heterocyclic group is also independently optionally substituted on a ring nitrogen atom with alkyl, aralkyl, alkylcarbonyl, or on sulfur with lower alkyl. Examples of heterocyclic groups include, without limitation, epoxy, azetidinyl, aziridinyl, tetrahydrofuranyl, tetrahydropyranyl, pyrrolidinyl, pyrrolidinonyl, piperidinyl, piperazinyl, imidazolidinyl, imidazopyridinyl, thiazolidinyl, dithianyl, trithianyl, dioxolanyl, oxazolidinyl, oxazolidinonyl, decahydroquinolinyl, piperidonyl, 4-piperidinonyl, quinuclidinyl, thiomorpholinyl, thiomorpholinyl 1,1 dioxide, morpholinyl, azepanyl, oxazepanyl, azabicyclohexanyls, azabicycloheptanyl, azabicyclooctanyls, azabicyclononanyls (e.g., octahydroindolizinyl), azaspiroheptanyls, dihydro-1H,3H,5H-oxazolo[3,4-c]oxazolyl, tetrahydro-1′H,3′H-spiro[cyclopropane-1,2′-pyrrolizine], hexahydro-1H-pyrrolizinyl, hexahydro-1H-pyrrolo[2,1-c][1,4]oxazinyl, octahydroindolizinyl, oxaazaspirononanyls, oxaazaspirooctanyls, diazaspirononanyls, oxaazabiocycloheptanyls, hexahydropyrrolizinyl 4(1H)-oxide, tetrahydro-2H-thiopyranyl 1-oxide and tetrahydro-2H-thiopyranyl 1,1-dioxide. Specifically excluded from the scope of this term are compounds having adjacent annular O and/or S atoms.

As used herein, the term “heteroaryl” refers to groups having 5 to 14 ring atoms, preferably 5, 6, 9, or 10 ring atoms; having 6, 10, or 14 π electrons shared in a cyclic array; and having, in addition to carbon atoms, from one to three heteroatoms per ring, or from one to three heteroatoms in at least one ring, selected from the group consisting of N, O, and S. Examples of heteroaryl groups include acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, 6,7-dihydro-5H-pyrrolo[1,2-a]imidazole, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. “Heteroaryl” also refers to bicyclic ring systems having, in addition to carbon atoms, from one to three heteroatoms per ring selected from the group consisting of N, O, and S in which one ring system may be saturated or partially saturated.

As used herein, “an effective amount” of a compound is an amount that is sufficient to negatively modulate or inhibit the activity of one or more of wild type KRas, KRas G12A, KRas G12C, KRas G12D, KRas G12R, KRas G12S, KRas G12V, KRas G13D or KRas Q61H. Such amount may be administered as a single dosage or may be administered according to a regimen, whereby it is effective.

As used herein, a “therapeutically effective amount” of a compound is an amount that is sufficient to ameliorate, or in some manner reduce a symptom or stop or reverse progression of a condition, or negatively modulate or inhibit the activity of one or more of wild type KRas, KRas G12A, KRas G12C, KRas G12D, KRas G12R, KRas G12S, KRas G12V, KRas G13D or KRas Q61H. Such amount may be administered as a single dosage or may be administered according to a regimen, whereby it is effective.

As used herein, treatment means any manner in which the symptoms or pathology of a condition, disorder or disease are ameliorated or otherwise beneficially altered. Treatment also encompasses any pharmaceutical use of the compositions herein.

As used herein, amelioration of the symptoms of a particular disorder by administration of a particular pharmaceutical composition refers to any lessening, whether permanent or temporary, lasting or transient that can be attributed to or associated with administration of the composition.

### Compounds

In one aspect of the invention, compounds are provided represented by Formula (I):

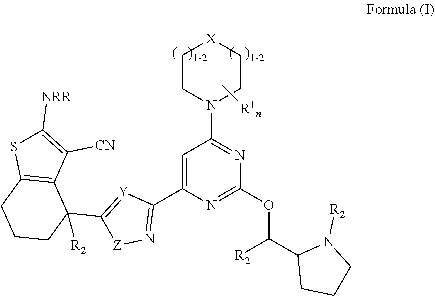

or a pharmaceutically acceptable salt thereof, wherein:
X is CR, O or N;
Y is CR or N;
Z is O or S;
n is an integer from 1 to 4;
each R is independently H or C1-C3 alkyl;
R¹ is C1-C3 alkyl or hydroxy; or
n is at least two, two and R¹s optionally join to form a methylene or ethylene bridge; or
n is at least two, and two R¹s optionally join to form a spiro or fused ring, where the ring is heterocyclic or heteroaryl, and where the ring is optionally substituted with 1-2 substituents selected from oxo and —C(O)N(CH3)(CH3);
each R² is independently C1-C3 alkyl.

In certain embodiments of the invention, n is 2 and two R¹s form a saturated heterocyclic ring containing S and N atoms.

In certain embodiments of the invention, the saturated heterocyclic ring formed by two R¹s is substituted with two oxos.

In certain embodiments of the invention, n is 2; and one R¹ is OH and the other R¹ is CH₃.

Non-limiting examples of compounds of Formula (I) are selected from the group consisting of:

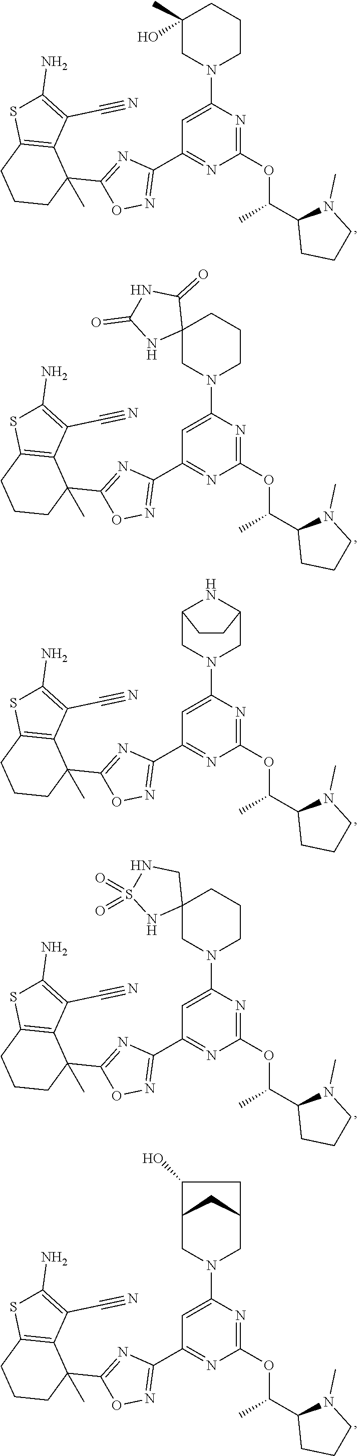 [SMILES: C[C@H](Oc1nc(-c2noc(C3(C)CCCc4sc(N)c(C#N)c43)n2)cc(N2CCC[C@@](C)(O)C2)n1)[C@@H]1CCCN1C ; C[C@H](Oc1nc(-c2noc(C3(C)CCCc4sc(N)c(C#N)c43)n2)cc(N2CCCC3(C2)NC(=O)NC3=O)n1)[C@@H]1CCCN1C ; C[C@H](Oc1nc(-c2noc(C3(C)CCCc4sc(N)c(C#N)c43)n2)cc(N2CC3CCC(C2)N3)n1)[C@@H]1CCCN1C ; C[C@H](Oc1nc(-c2noc(C3(C)CCCc4sc(N)c(C#N)c43)n2)cc(N2CCCC3(CNS(=O)(=O)N3)C2)n1)[C@@H]1CCCN1C ; C[C@H](Oc1nc(-c2noc(C3(C)CCCc4sc(N)c(C#N)c43)n2)cc(N2C[C@@H]3C[C@H](C2)[C@H](O)C3)n1)[C@@H]1CCCN1C]

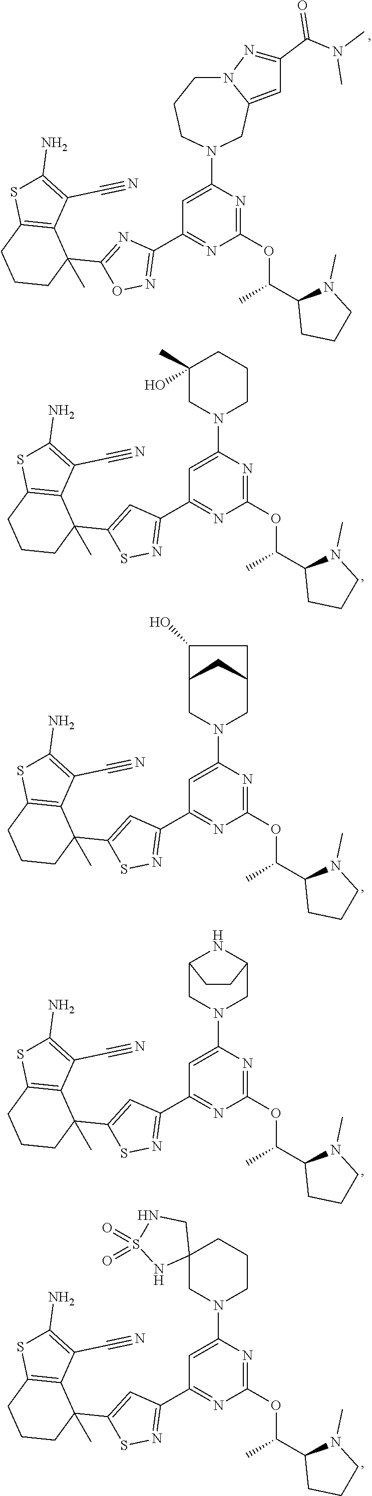 [SMILES: C[C@H](Oc1nc(-c2noc(C3(C)CCCc4sc(N)c(C#N)c43)n2)cc(N2CCCn3nc(C(=O)N(C)C)cc3C2)n1)[C@@H]1CCCN1C ; C[C@H](Oc1nc(-c2cc(C3(C)CCCc4sc(N)c(C#N)c43)sn2)cc(N2CCC[C@@](C)(O)C2)n1)[C@@H]1CCCN1C ; C[C@H](Oc1nc(-c2cc(C3(C)CCCc4sc(N)c(C#N)c43)sn2)cc(N2C[C@@H]3C[C@H](C2)[C@H](O)C3)n1)[C@@H]1CCCN1C ; C[C@H](Oc1nc(-c2cc(C3(C)CCCc4sc(N)c(C#N)c43)sn2)cc(N2CC3CCC(C2)N3)n1)[C@@H]1CCCN1C ; C[C@H](Oc1nc(-c2cc(C3(C)CCCc4sc(N)c(C#N)c43)sn2)cc(N2CCCC3(CNS(=O)(=O)N3)C2)n1)[C@@H]1CCCN1C]

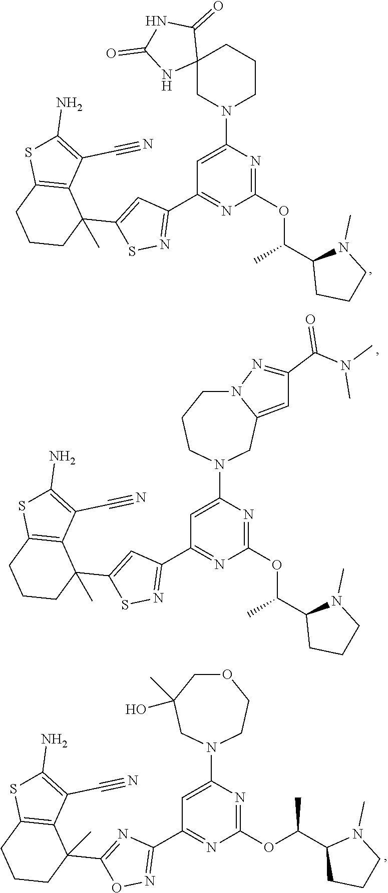

and pharmaceutically acceptable salts thereof.

In one embodiment, the compounds of Formula (I) include bis-hydrochloride, tris-hydrochloride, trifluoroacetic acid, bis-trifluoroacetic acid, and tris-trifluoracetic acid salts of the above compounds. The compounds of Formula (I) or pharmaceutically acceptable salt thereof may be formulated into pharmaceutical compositions.

### Pharmaceutical Compositions

In another aspect, the invention provides pharmaceutical compositions comprising a wild type KRas, KRas G12A, KRas G12C, KRas G12D, KRas G12R, KRas G12S, KRas G12V, KRas G13D and/or KRas Q61H inhibitor according to the invention and a pharmaceutically acceptable carrier, excipient, or diluent. Compounds of the invention may be formulated by any method well known in the art and may be prepared for administration by any route, including, without limitation, parenteral, intraperitoneal, intradermal, intracardiac, intraventricular, intracranial, intracerebrospinal, intrasynovial, intrathecal administration, intramuscular injection, intravitreous injection, intravenous injection, intra-arterial injection, oral, buccal, sublingual, transdermal, topical, intranasal, intratracheal, intrarectal, subcutaneous, and topical administration. In certain embodiments, compounds of the invention are administered intravenously in a hospital setting. In one embodiment, administration may be by the oral route. In some embodiments, the provided pharmaceutical compositions may be administered to a subject in need of treatment by injection systemically, such as by intravenous injection; or by injection or application to the relevant site, such as by direct injection via syringe, or direct application to the site when the site is exposed in surgery; or by topical administration.

Parenteral administration can be by bolus injection or continuous infusion. Pharmaceutical compositions for injection may be presented in unit dosage form, e.g., in ampoules or in multi-dose containers, with an added preservative.

The provided pharmaceutical compositions can also be formulated as a depot preparation. Such long acting formulations may be administered by implantation (for example subcutaneously or intramuscularly) or by intramuscular injection. Thus, for example, the formulations may be modified with suitable polymeric or hydrophobic materials (for example as an emulsion in an acceptable oil) or ion exchange resins, or as sparingly soluble derivatives, for example, as a sparingly soluble salt.

The pharmaceutical compositions may, if desired, be presented in a vial, pack or a medical device, including but not limited to a dispenser device which may contain one or more unit dosage forms containing the active ingredient. In one embodiment the dispenser device can comprise a syringe having a single dose of the liquid formulation ready for injection. The syringe can be accompanied by instructions for administration.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term pharmaceutically acceptable salt refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. In one embodiment, a dose of the active compound for all of the above-mentioned conditions is in the range from about 0.01 to 300 mg/kg, for example 0.1 to 100 mg/kg per day, and as a further example 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising compounds of the present invention may be used in the methods of use described herein.

### METHODS OF USE

In yet another aspect, the invention provides for methods for inhibiting wild type KRas, KRas G12A, KRas G12C, KRas G12D, KRas G12R, KRas G12S, KRas G12V and/or KRas Q61H activity in a cell, comprising contacting the cell in which inhibition of wild type KRas, KRas G12A, KRas G12C, KRas G12D, KRas G12R, KRas G12S, KRas G12V and/or Q61H activity is desired with an effective amount of a compound of Formula (I), pharmaceutically acceptable salts thereof, or pharmaceutical compositions containing the compound or pharmaceutically acceptable salt thereof. In one embodiment, the contacting is in vitro. In one embodiment, the contacting is in vivo.

As used herein, the term “contacting” refers to the bringing together of indicated moieties in an in vitro system or an in vivo system. For example, “contacting” wild type KRas, KRas G12A, KRas G12C, KRas G12D, KRas G12R, KRas G12S, KRas G12V, KRas G13D and/or KRas Q61H with a compound provided herein includes the administration of a compound provided herein to an individual or patient, such as a human, having wild type KRas or a KRas G12A, KRas G12C, KRas G12D, KRas G12R, KRas G12S, KRas G12V, KRas G13D and/or KRas Q61H mutation, as well as, for example, introducing a compound provided herein into a sample containing a cellular or purified preparation containing wild type KRas or a KRas G12A, KRas G12C, KRas G12D, KRas G12R, KRas G12S, KRas G12V, KRas G13D or KRas Q61H mutation.

In one embodiment, a cell in which inhibition of wild type KRas or KRas G12A, KRas G12C, KRas G12D, KRas G12R, KRas G12S, KRas G12V, KRas G13D and/or KRas Q61H activity is desired is contacted with an effective amount of a compound of Formula (I) or pharmaceutically acceptable salt thereof to negatively modulate the activity of one or more of wild type KRas or KRas G12A, KRas G12C, KRas G12D, KRas G12R, KRas G12S, KRas G12V, KRas G13D and KRas Q61H.

By negatively modulating the activity of one or more of wild type KRas or KRas G12A, KRas G12C, KRas G12D, KRas G12R, KRas G12S, KRas G12V, KRas G13D and KRas Q61H, the methods described herein are designed to inhibit undesired cellular proliferation resulting from enhanced wild type KRas or KRas G12A, KRas G12C, KRas G12D, KRas G12R, KRas G12S, KRas G12V, KRas G13D and/or KRas Q61H activity within the cell. The cells may be contacted in a single dose or multiple doses in accordance with a particular treatment regimen to affect the desired negative modulation of wild type KRas or KRas G12A, KRas G12C, KRas G12D, KRas G12R, KRas G12S, KRas G12V, KRas G13D and/or KRas Q61H. The ability of compounds to bind one or more of wild type KRas or KRas G12A, KRas G12C, KRas G12D, KRas G12R, KRas G12S, KRas G12V, KRas G13D and KRas Q61H may be monitored in vitro using well known methods, including those described in Examples A and B below. In addition, the inhibitory activity of exemplary compounds in cells may be monitored, for example, by measuring the inhibition of one or more of wild type KRas or KRas G12A, KRas G12C, KRas G12D, KRas G12R, KRas G12S, KRas G12V, KRas G13D and/or KRas Q61H activity of the amount of phosphorylated ERK, for example using the method described in Example C below.

In another aspect, methods of treating cancer in a patient in need thereof, comprising administering to said patient a therapeutically effective amount of a compound of Formula (I) or a pharmaceutically acceptable salt thereof, or a pharmaceutical composition comprising the compound or pharmaceutically acceptable salt thereof are provided.

The compositions and methods provided herein may be used for the treatment of a wild type KRas-associated or KRas G12A, KRas G12C, KRas G12D, KRas G12R, KRas G12S, KRas G12V, KRas G13D and/or KRas Q61H-associated cancer in a patient in need thereof, comprising administering to said patient a therapeutically effective amount of a compound of Formula (I), a pharmaceutically acceptable salt thereof, or a pharmaceutical composition comprising the compound or pharmaceutically acceptable salt thereof are provided. In one embodiment, the wild type KRas-associated or KRas G12A, KRas G12C, KRas G12D, KRas G12R, KRas G12S, KRas G12V, KRas G13D and/or KRas Q61H-associated cancer is lung cancer.

The compositions and methods provided herein may be used for the treatment of a wide variety of cancers including tumors such as lung, prostate, breast, brain, skin, cervical carcinomas, testicular carcinomas, etc. More particularly, cancers that may be treated by the compositions and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is non-small cell lung cancer, small cell lung cancer, colorectal cancer, rectal cancer or pancreatic cancer. In certain embodiments, the cancer is non-small cell lung cancer.

The concentration and route of administration to the patient will vary depending on the cancer to be treated. The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

Also provided herein is a compound of Formula (I) or a pharmaceutically acceptable salt thereof, or a pharmaceutical composition thereof as defined herein for use in therapy.

Also provided herein is a compound of Formula (I) or a pharmaceutically acceptable salt thereof, or a pharmaceutical composition thereof as defined herein for use in the treatment of cancer.

Also provided herein is a compound of Formula (I) or a pharmaceutically acceptable salt thereof, or a pharmaceutical composition thereof for use in the inhibition of wild type KRas or KRas G12A, KRas G12C, KRas G12D, KRas G12R, KRas G12S, KRas G12V, KRas G13D and/or KRas Q61H.

Also provided herein is a compound of Formula (I) or a pharmaceutically acceptable salt thereof, or a pharmaceutical composition thereof as defined herein, for use in the treatment of wild type KRas-associated or a KRas G12A, KRas G12C, KRas G12D, KRas G12R, KRas G12S, KRas G12V, KRas G13D and/or KRas Q61H-associated disease or disorder.

Also provided herein is the use of a compound of Formula (I) or a pharmaceutically acceptable salt thereof, as defined herein in the manufacture of a medicament for the treatment of cancer.

Also provided herein is a use of a compound of Formula (I) or a pharmaceutically acceptable salt thereof, as defined herein in the manufacture of a medicament for the inhibition of activity of wild type KRas or KRas G12A, KRas G12C, KRas G12D, KRas G12R, KRas G12S, KRas G12V, KRas G13D and/or KRas Q61H.

Also provided herein is the use of a compound of Formula (I) or a pharmaceutically acceptable salt thereof, as defined herein, in the manufacture of a medicament for the treatment of wild type KRas-associated or a KRas G12A, KRas G12C, KRas G12D, KRas G12R, KRas G12S, KRas G12V, KRas G13D and/or KRas Q61H-associated disease or disorder.

Also provided herein is a method for treating cancer in a patient in need thereof, the method comprising (a) determining that cancer is associated with wild type KRas or a KRas G12A, KRas G12C, KRas G12D, KRas G12R, KRas G12S, KRas G12V, KRas G13D and/or KRas Q61H mutation (e.g., as determined using a regulatory agency-approved, e.g., FDA-approved, assay or kit); and (b) administering to the patient a therapeutically effective amount of a compound of Formula (I), or a pharmaceutically acceptable salt thereof, or a pharmaceutical composition thereof.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

### Reaction Schemes and Examples

The compounds of the present invention may be prepared from commercially available reagents using the synthetic methods and reaction schemes described herein, or using other reagents and conventional methods well known to those skilled in the art. For instance, compounds of the present invention may be prepared according to the reaction schemes and examples outlines below.

The compounds of the present invention may have one or more chiral center and may be synthesized as stereoisomeric mixtures, isomers of identical constitution that differ in the arrangement of their atoms in space. The compounds may be used as mixtures or the individual components/isomers may be separated using commercially available reagents and conventional methods for isolation of stereoisomers and enantiomers well-known to those skilled in the art, e.g., using CHIRALPAK® (Sigma-Aldrich) or CHIRALCEL® (Diacel Corp) chiral chromatographic HPLC columns according to the manufacturer's instructions. Alternatively, compounds of the present invention may be synthesized using optically pure, chiral reagents and intermediates to prepare individual isomers or enantiomers. Unless otherwise indicated, all chiral (enantiomeric and diastereomeric) and racemic forms are within the scope of the invention. Unless otherwise indicated, whenever the specification, including the claims, refers to compounds of the invention, the term “compound” is to be understood to encompass all chiral (enantiomeric and diastereomeric) and racemic forms.

The compounds of the present invention may be in anhydrous, solvated or hydrated forms, and all such forms are included within the scope of the invention.

The following Intermediates are intended to illustrate further certain embodiments of the invention and are not intended to limit the scope of the invention.

### Example 1

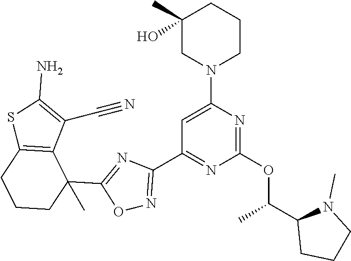 [SMILES: C[C@H](Oc1nc(-c2noc(C3(C)CCCc4sc(N)c(C#N)c43)n2)cc(N2CCC[C@@](C)(O)C2)n1)[C@@H]1CCCN1C]

### 2-amino-4-(3-(6-((R)-3-hydroxy-3-methylpiperidin-1-yl)-2-((S)-1-((S)-1-methylpyrrolidin-2-yl)ethoxy)pyrimidin-4-yl)-1,2,4-oxadiazol-5-yl)-4-methyl-4,5,6,7-tetrahydrobenzo[b]thiophene-3-carbonitrile

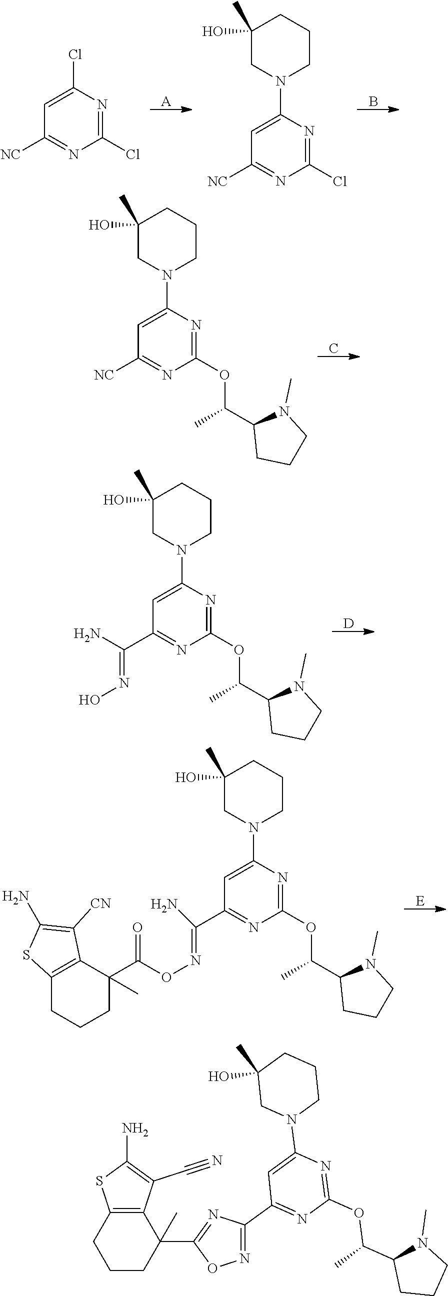

Step A. (R)-2-chloro-6-(3-hydroxy-3-methylpiperidin-1-yl)pyrimidine-4-carbonitrile: To a solution of 2,6-dichloropyrimidine-4-carbonitrile (2.00 g, 11.5 mmol, 1.00 eq) in DCM (20.0 mL) was added dropwise DIEA (2.97 g, 23.0 mmol, 4.00 mL, 2.00 eq) at 0° C. Then (3R)-3-methylpiperidin-3-ol (1.74 g, 11.5 mmol, 1.00 eq, HCl) was added to the mixture at 0° C. The resulting mixture was stirred at 0° C. for 2 hrs. The reaction mixture was with H₂O (10.0 mL) at 0° C., and then extracted with DCM 15.0 mL (10.0 mL×3). The combined organic layers were washed with brine (20.0 mL), dried over Na₂SO₄, filtered, and concentrated under reduced pressure to give a residue. The residue was purified by column chromatography (SiO₂, petroleum ether/ethyl acetate 20:1 to 1:1) to give the title compound (1.70 g, 6.66 mmol, 57.9% yield, 99.0% purity) as a yellow solid. ¹HNMR: (400 MHZ, CDCl₃) δ 6.83 (s, 1H), 4.60-3.50 (m, 2H), 3.20-3.12 (m, 2H), 2.00-1.65 (m, 2H), 1.64-1.51 (m, 2H), 1.33 (s, 3H).

Step B. 6-((R)-3-hydroxy-3-methylpiperidin-1-yl)-2-((S)-1-((S)-1-methylpyrrolidin-2-yl)ethoxy)pyrimidine-4-carbonitrile: To a solution of (R)-2-chloro-6-(3-hydroxy-3-methylpiperidin-1-yl)pyrimidine-4-carbonitrile (500 mg, 1.98 mmol, 1.00 eq) in ACN (5.00 mL) was added dropwise DIPEA (767 mg, 5.94 mmol, 1.03 mL, 3.00 eq) and(S)-1-((S)-1-methylpyrrolidin-2-yl) ethan-1-ol (383 mg, 2.97 mmol, 1.50 eq). The reaction was stirred at 80° C. for 14 hrs. Another batch of(S)-1-((S)-1-methylpyrrolidin-2-yl) ethan-1-ol (256 mg, 1.98 mmol, 1.00 eq) was added to the reaction. The resulting mixture was stirred at 80° C. for 16 hrs. The reaction mixture was quenched with water (10.0 mL) and extracted with DCM (5.00 mL×3). The combined organic layers were washed with brine (10.0 mL) and dried over Na₂SO₄, filtered and concentrated under reduced pressure to give a residue. The residue was purified by column chromatography (SiO₂, petroleum ether/ethyl acetate=10:1 to DCM/MeOH 50:1) to give the title compound (400 mg, 1.07 mmol, 53.8% yield, 92.0% purity) as a yellow oil. ¹H NMR: (400 MHZ, CDCl3) δ 6.59 (s, 1H), 5.30-5.19 (m, 1H), 3.80-3.70 (m, 1H), 3.50-3.30 (m, 1H), 3.25-3.05 (m, 3H), 2.74-2.60 (m, 1H), 2.48 (s, 3H), 2.30-2.25 (m, 1H), 2.10-1.75 (m, 8H), 1.60-1.50 (m, 3H), 1.30-1.25 (m, 6H).

Step C. (Z)—N′-hydroxy-6-((R)-3-hydroxy-3-methylpiperidin-1-yl)-2-((S)-1-((S)-1-methylpyrrolidin-2-yl)ethoxy)pyrimidine-4-carboximidamide: To a solution of 6-((R)-3-hydroxy-3-methylpiperidin-1-yl)-2-((S)-1-((S)-1-methylpyrrolidin-2-yl)ethoxy)pyrimidine-4-carbonitrile (500 mg, 1.45 mmol, 1.00 eq) in EtOH (5.00 mL) were added Na₂CO₃ (199 mg, 1.88 mmol, 1.30 eq) and NH₂OH·HCl (262 mg, 3.76 mmol, 2.60 eq). The resulting solution was stirred at 80° C. for 1 hr. The reaction mixture was filtered and the filtrate was concentrated under reduced pressure to give the title compound (540 mg, crude) as a yellow solid. LCMS (ESI): m/z=379.3 (M+1)⁺;

Step D. (Z)—N′-((2-amino-3-cyano-4-methyl-4,5,6,7-tetrahydrobenzo[b]thiophene-4-carbonyl)oxy)-6-((R)-3-hydroxy-3-methylpiperidin-1-yl)-2-((S)-1-((S)-1-methylpyrrolidin-2-yl)ethoxy)pyrimidine-4-carboximidamide: To a solution of (Z)—N′-hydroxy-6-((R)-3-hydroxy-3-methylpiperidin-1-yl)-2-((S)-1-((S)-1-methylpyrrolidin-2-yl)ethoxy)pyrimidine-4-carboximidamide (120 mg, 317 μmol, 1.00 eq) and 2-amino-3-cyano-4-methyl-4,5,6,7-tetrahydrobenzo[b]thiophene-4-carboxylic acid (82.4 mg, 349 μmol, 1.10 eq) in DMF (1.00 mL) was added DIEA (123 mg, 951 μmol, 165.68 μL, 3.00 eq) and PyBOP (248 mg, 476 μmol, 1.50 eq) at 0° C. The result solution was stirred at 0° C. to rt for 1 hr. The reaction was concentrated and purified by pre-HPLC (column: Waters Xbridge 150*25 mm*5 um; mobile phase: [water (NH₄HCO₃)-ACN]; B %: 38%-68%, 8 min) to give the title compound (100 mg, 153 μmol, 48.2% yield, 91.2% purity) as a yellow solid. LCMS (ESI): m/z=597.3 (M+1)⁺.

Step E. 2-amino-4-(3-(6-((R)-3-hydroxy-3-methylpiperidin-1-yl)-2-((S)-1-((S)-1-methylpyrrolidin-2-yl)ethoxy)pyrimidin-4-yl)-1,2,4-oxadiazol-5-yl)-4-methyl-4,5,6,7-tetrahydrobenzo[b]thiophene-3-carbonitrile: To a solution of (Z)—N′-((2-amino-3-cyano-4-methyl-4,5,6,7-tetrahydrobenzo[b]thiophene-4-carbonyl)oxy)-6-((R)-3-hydroxy-3-methylpiperidin-1-yl)-2-((S)-1-((S)-1-methylpyrrolidin-2-yl)ethoxy)pyrimidine-4-carboximidamide (100 mg, 153 μmol, 91.2% purity, 1.00 eq) in THF (1.00 mL) was added Triton B (56.2 mg, 134 μmol, 61.1 μL, 40.0% purity, 0.9 eq) at 0° C. The result solution was stirred at 0° C. for 1 hr. The reaction was concentrated under reduced pressure. The residue was purified by pre-HPLC (column: Waters Xbridge 150*25 mm*5 um; mobile phase: [water (NH₄HCO₃)-ACN]; B %: 45%-75%, 8 min) and lyophilized to give the title compound (5.20 mg, 9.60 μmol, 6.28% yield, 92.6% purity) as a yellow solid. LCMS (ESI): m/z=579.3 (M+1)⁺; ¹H NMR (400 MHz, MeOD) δ 7.13 (s, 1H), 5.27-5.10 (m, 1H), 4.73-4.51 (m, 5H), 3.91-3.68 (m, 1H), 3.13 (s, 1H), 2.90-2.72 (m, 1H), 2.64-2.55 (m, 4H), 2.50-2.45 (m, 1H), 2.25-2.18 (m, 1H), 2.10-2.00 (m, 2H), 1.98-1.92 (m, 2H), 1.90-1.68 (m, 9H), 1.65-1.56 (m, 1H), 1.38-1.32 (m, 3H), 1.25 (s, 3H).

### Example 2

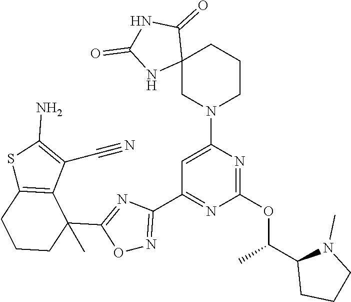 [SMILES: C[C@H](Oc1nc(-c2noc(C3(C)CCCc4sc(N)c(C#N)c43)n2)cc(N2CCCC3(C2)NC(=O)NC3=O)n1)[C@@H]1CCCN1C]

### 2-amino-4-(3-(6-(2,4-dioxo-1,3,7-triazaspiro[4.5]decan-7-yl)-2-((S)-1-((S)-1-methylpyrrolidin-2-yl)ethoxy)pyrimidin-4-yl)-1,2,4-oxadiazol-5-yl)-4-methyl-4,5,6,7-tetrahydrobenzo[b]thiophene-3-carbonitrile

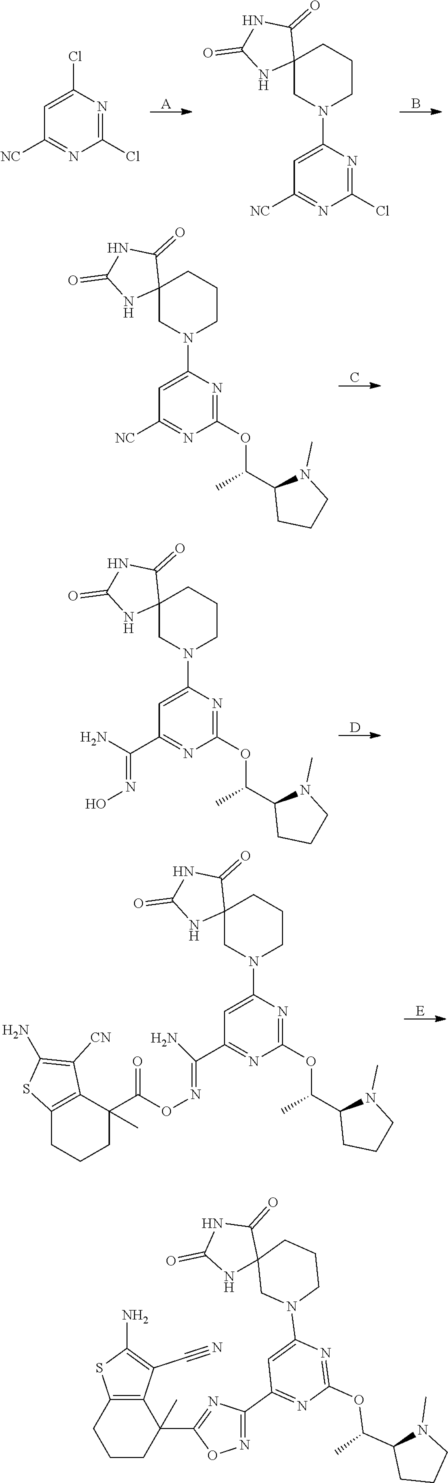

Step A to D were performed as Example 1 step A to D.

Step E. 2-amino-4-(3-(6-(2,4-dioxo-1,3,7-triazaspiro[4.5]decan-7-yl)-2-((S)-1-((S)-1-methylpyrrolidin-2-yl)ethoxy)pyrimidin-4-yl)-1,2,4-oxadiazol-5-yl)-4-methyl-4,5,6,7-tetrahydrobenzo[b]thiophene-3-carbonitrile: To a solution of (Z)—N′-((2-amino-3-cyano-4-methyl-4,5,6,7-tetrahydrobenzo[b]thiophene-4-carbonyl)oxy)-6-(2,4-dioxo-1,3,7-triazaspiro[4.5]decan-7-yl)-2-((S)-1-((S)-1-methylpyrrolidin-2-yl)ethoxy)pyrimidine-4-carboximidamide (110 mg, 149 μmol, 88.0% purity, 1.00 eq) in DMF (1.00 mL) was added K₂CO₃ (41.12 mg, 297.50 μmol, 2.00 eq). The mixture was stirred at 60 to 100° C. for 12 hrs. The reaction mixture was filtered, and then the organic layers was concentrated under reduced pressure to give a residue. The crude product was purified by reversed-phase HPLC (column: Waters Xbridge 150*25 mm*5 um; mobile phase: [water (NH₄HCO₃)-ACN]; B %: 29%-59%, 8 min) to give the title compound (15.0 mg, 22.2 μmol, 14.9% yield, 93.5% purity) as a yellow solid. LCMS (ESI): m/z=633.3 (M+1)⁺; ¹H NMR (400 MHZ, MeOD) δ 7.17 (s, 1H), 3.18-3.15 (m, 8H), 3.14 (s, 1H), 2.60 (s, 1H), 2.61 (m, 1H), 2.52 (m, 2H), 2.39-2.35 (m, 1H), 2.24-2.12 (m, 2H), 1.98-1.93 (m, 3H), 1.88-1.69 (m, 14H), 1.32 (d, J=6 Hz, 3H).

### Example 3

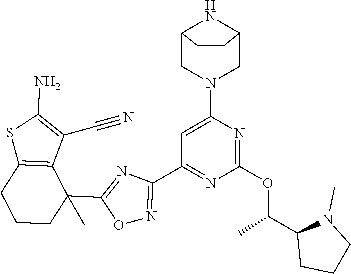 [SMILES: C[C@H](Oc1nc(-c2noc(C3(C)CCCc4sc(N)c(C#N)c43)n2)cc(N2CC3CCC(C2)N3)n1)[C@@H]1CCCN1C]

### 4-(3-(6-((1R,5S)-3,8-diazabicyclo[3.2.1]octan-3-yl)-2-((S)-1-((S)-1-methylpyrrolidin-2-yl)ethoxy)pyrimidin-4-yl)-1,2,4-oxadiazol-5-yl)-2-amino-4-methyl-4,5,6,7-tetrahydrobenzo[b]thiophene-3-carbonitrile

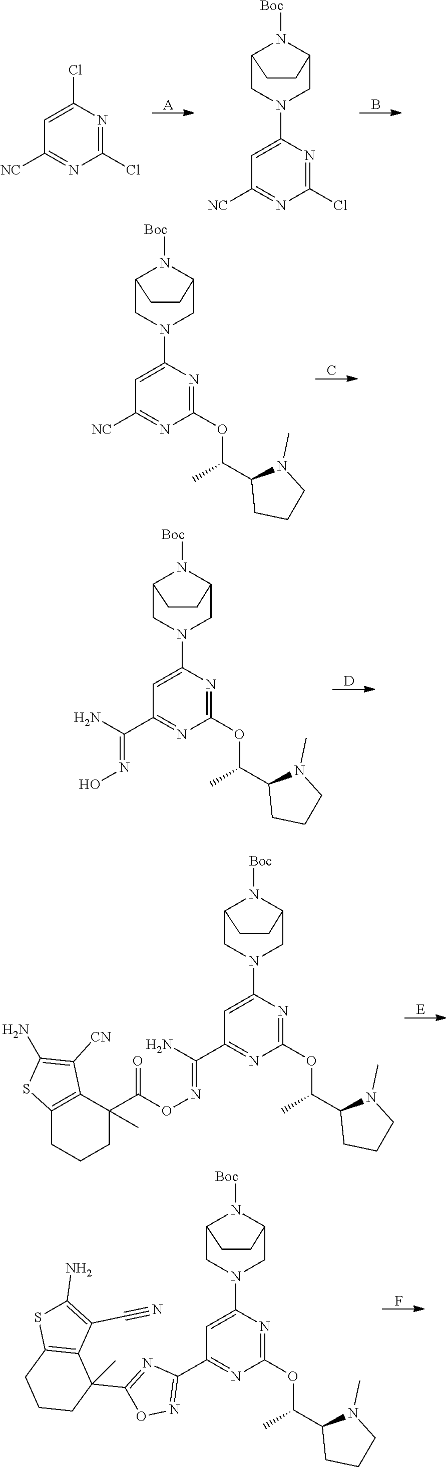

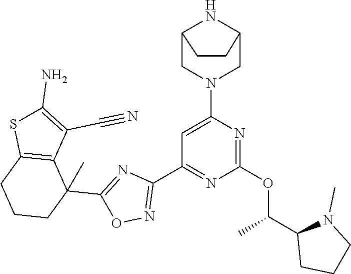 [SMILES: C[C@H](Oc1nc(-c2noc(C3(C)CCCc4sc(N)c(C#N)c43)n2)cc(N2CC3CCC(C2)N3)n1)[C@@H]1CCCN1C]

Step A to E were performed as Example 1 step A to E.

Step F. 4-(3-(6-((1R,5S)-3,8-diazabicyclo[3.2.1]octan-3-yl)-2-((S)-1-((S)-1-methylpyrrolidin-2-yl)ethoxy)pyrimidin-4-yl)-1,2,4-oxadiazol-5-yl)-2-amino-4-methyl-4,5,6,7-tetrahydrobenzo[b]thiophene-3-carbonitrile: To a solution of tert-butyl (1R,5S)-3-(6-(5-(2-amino-3-cyano-4-methyl-4,5,6,7-tetrahydrobenzo[b]thiophen-4-yl)-1,2,4-oxadiazol-3-yl)-2-((S)-1-((S)-1-methylpyrrolidin-2-yl)ethoxy)pyrimidin-4-yl)-3,8-diazabicyclo[3.2.1]octane-8-carboxylate (20.0 mg, 28.7 μmol, 97.0% purity, 1.00 eq) in dioxane (1.00 mL) was added HCl/dioxane (4.0 M, 0.50 mL, 92.9 eq). The mixture was stirred at 25° C. for 4 hrs. The reaction mixture was concentrated under reduced pressure. The residue was diluted with water (4.00 mL) and extracted with DCM (4.00 mL×3). The pH of aqueous phase was adjusted to 8 with solid NaHCO₃ and extracted with DCM (4.00 mL×4). The combined organic layers were washed with brine (4.00 mL×2), dried over Na₂SO₄, filtered, and concentrated to give a residue. ACN/H2O (1/5, 20V) was added and triturated at 25° C. for 1 hr, then freeze-dried to give the title compound (10.0 mg, 15.8 μmol, 54.9% yield) as a yellow solid. LCMS (ESI): m/z=576.3 (M+1)⁺; ¹H NMR (400 MHZ, MeOD) δ 7.08 (s, 1H), 5.27-5.21 (m, 1H), 3.68-3.66 (m, 2H), 3.31-3.30 (m, 1H), 3.29-3.28 (m, 1H), 3.06 (m, 1H). 2.69-2.62 (m, 3H), 2.61 (m, 2H), 2.22-2.19 (m, 2H), 2.00-1.79 (m, 11H), 1.76 (m, 2H), 1.40-1.37 (m, 3H), 1.33-1.29 (m, 4H).

### Example 4

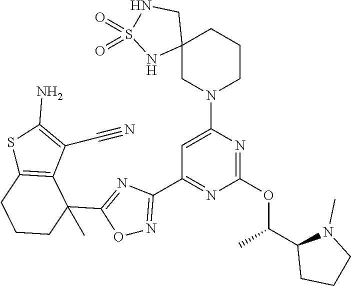 [SMILES: C[C@H](Oc1nc(-c2noc(C3(C)CCCc4sc(N)c(C#N)c43)n2)cc(N2CCCC3(CNS(=O)(=O)N3)C2)n1)[C@@H]1CCCN1C]

### 2-amino-4-(3-(6-(2,2-dioxido-2-thia-1,3,7-triazaspiro[4.5]decan-7-yl)-2-((S)-1-((S)-1-methylpyrrolidin-2-yl)ethoxy)pyrimidin-4-yl)-1,2,4-oxadiazol-5-yl)-4-methyl-4,5,6,7-tetrahydrobenzo[b]thiophene-3-carbonitrile

Synthesized according to Example 2. The title compound was obtained as yellow solid. LCMS: M/Z=655.4 [M+1]⁺. ¹H NMR: 400 MHZ MeOD-d⁴, δ 8.52 (s, 1H), 7.28-7.26 (m, 1H), 5.34-5.33 (m, 1H), 4.05-3.99 (m, 1H), 3.71-3.59 (m, 3H), 3.50-3.34 (m, 3H), 3.21-3.13 (m, 2H), 3.03-2.99 (m, 3H), 2.63 (t, J=6.00 Hz 2H), 2.37-2.34 (m, 1H), 2.26-2.09 (m, 3H), 2.04-1.92 (m, 6H), 1.90 (s, 3H), 1.84-1.78 (m, 2H), 1.48-1.47 (d, J=6.00 Hz 3H).

### Example 5

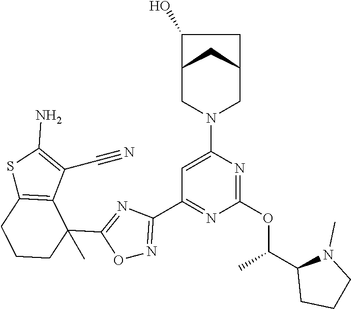 [SMILES: C[C@H](Oc1nc(-c2noc(C3(C)CCCc4sc(N)c(C#N)c43)n2)cc(N2C[C@@H]3C[C@H](C2)[C@H](O)C3)n1)[C@@H]1CCCN1C]

### 2-amino-4-(3-(6-((1R,5R,6R)-6-hydroxy-3-azabicyclo[3.2.1]octan-3-yl)-2-((S)-1-((S)-1-methylpyrrolidin-2-yl)ethoxy)pyrimidin-4-yl)-1,2,4-oxadiazol-5-yl)-4-methyl-4,5,6,7-tetrahydrobenzo[b]thiophene-3-carbonitrile

Synthesized according to Example 2. The title compound was obtained as yellow solid (500 mg, 0.804 mmol, 30.5% yield, 94.3% purity, formic acid salt). LCMS (ESI): m/z=591.3 [M+1]⁺. ¹H NMR (400 MHZ MeOD-d⁴) δ 8.53 (s, 1H), 7.17-7.15 (m, 1H), 5.27 (s, 1H), 4.76-4.47 (m, 2H), 4.33-4.30 (m, 1H), 4.20-4.18 (m, 1H), 3.69-3.60 (m, 2H), 3.16-3.08 (m, 3H), 3.07-3.05 (m, 3H), 2.81-2.61 (m, 2H), 2.27-2.22 (m, 2H), 2.19-2.08 (m, 4H), 2.06-1.98 (m, 5H), 1.90 (s, 2H), 1.85-1.75 (m, 2H), 1.49-1.44 (m, 3H), 1.24-1.21 (m, 1H).

### Example 6

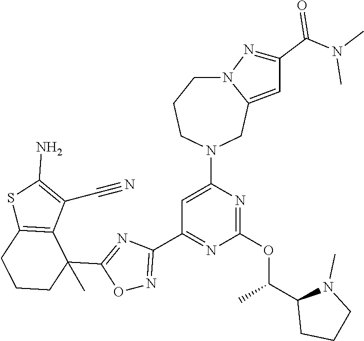 [SMILES: C[C@H](Oc1nc(-c2noc(C3(C)CCCc4sc(N)c(C#N)c43)n2)cc(N2CCCn3nc(C(=O)N(C)C)cc3C2)n1)[C@@H]1CCCN1C]

### 5-(6-(5-(2-amino-3-cyano-4-methyl-4,5,6,7-tetrahydrobenzo[b]thiophen-4-yl)-1,2,4-oxadiazol-3-yl)-2-((S)-1-((S)-1-methylpyrrolidin-2-yl)ethoxy)pyrimidin-4-yl)-N,N-dimethyl-5,6,7,8-tetrahydro-4H-pyrazolo[1,5-a][1,4]diazepine-2-carboxamide

Synthesized according to Example 2. The title compound was obtained as white solid (15.4 mg, 21.6 μmol, 14.9% yield, 94.5% purity, formic acid salt). LCMS (ESI): m/z=672.3 [M+1]⁺. ¹H NMR (400 MHZ MeOD-d⁴) δ 8.53 (s, 1H), 7.23 (s, 1H), 6.68 (s, 1H), 5.24 (s, 1H), 4.78 (m, 8H), 4.10 (m, 1H), 3.31-3.30 (m, 2H), 3.05 (s, 3H), 2.85 (m, 4H), 2.61 (t, J=4.00 Hz, 2H), 2.23 (m, 2H), 2.09-1.85 (m, 11H), 1.44 (s, 3H).

### Example 7

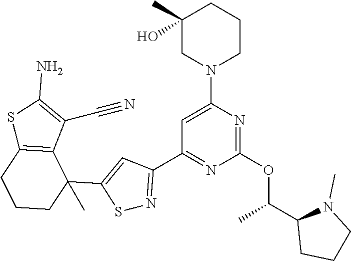 [SMILES: C[C@H](Oc1nc(-c2cc(C3(C)CCCc4sc(N)c(C#N)c43)sn2)cc(N2CCC[C@@](C)(O)C2)n1)[C@@H]1CCCN1C]

### 2-amino-4-(3-(6-((R)-3-hydroxy-3-methylpiperidin-1-yl)-2-((S)-1-((S)-1-methylpyrrolidin-2-yl)ethoxy)pyrimidin-4-yl) isothiazol-5-yl)-4-methyl-4,5,6,7-tetrahydrobenzo[b]thiophene-3-carbonitrile

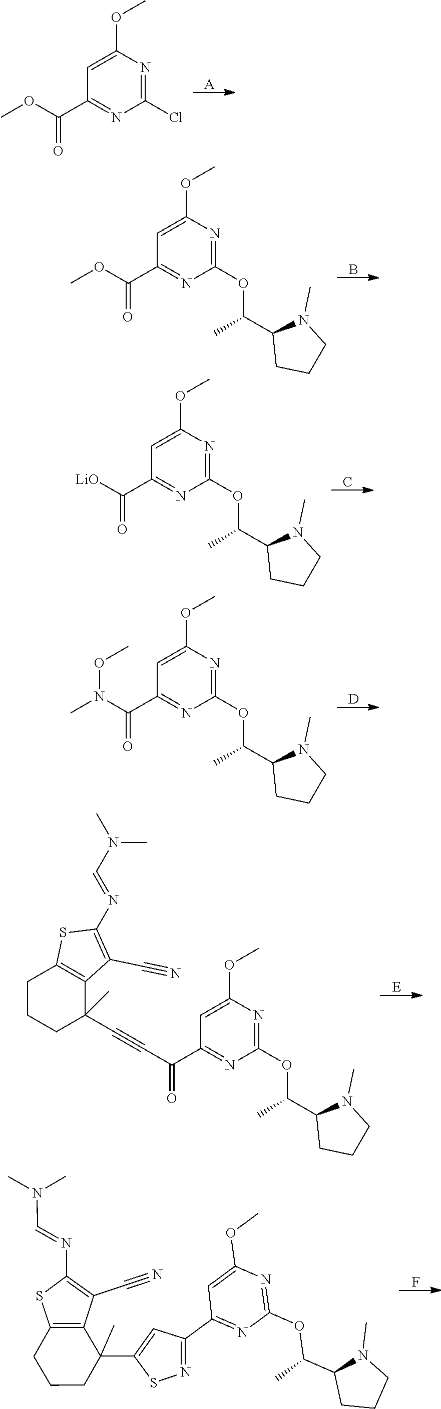

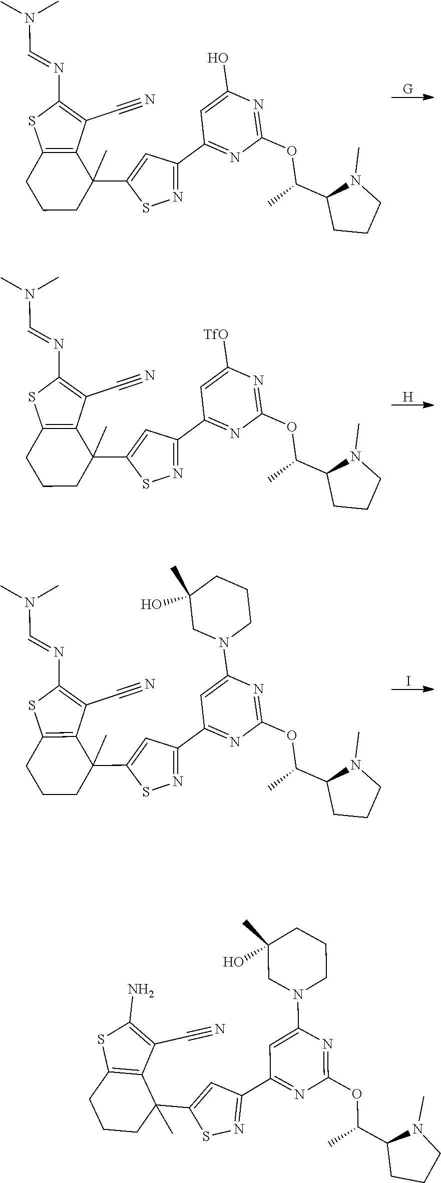

Step A: methyl 6-methoxy-2-((S)-1-((S)-1-methylpyrrolidin-2-yl)ethoxy)pyrimidine-4-carboxylate. To a mixture of methyl 2-chloro-6-methoxypyrimidine-4-carboxylate (20.0 g, 98.7 mmol) and(S)-1-((S)-1-methylpyrrolidin-2-yl) ethan-1-ol (15.3 g, 118 mmol) in ACN (200 mL) was added DIPEA (25.5 g, 197 mmol, 34.3 mL) in one portion at 20° C. under N₂. The reaction was heated to 80° C. and stirred for 40 hrs. The residue was poured into water (200 mL). The aqueous mixture was extracted with dichloromethane (100 mL×2). The combined organic phases were washed with brine (100 mL), dried with anhydrous Na₂SO₄, filtered, concentrated and purified by column chromatography (SiO₂, Petroleum ether:Ethyl acetate=10:1˜1:0) to give the title compound as yellow solid (18.0 g, 48.7 mmol, 49.4% yield, 80.0% purity). LCMS: m/z=296.2 (M+1)⁺.

Step B: lithium 6-methoxy-2-((S)-1-((S)-1-methylpyrrolidin-2-yl)ethoxy)pyrimidine-4-carboxylate. To a solution of methyl 6-methoxy-2-((S)-1-((S)-1-methylpyrrolidin-2-yl)ethoxy)pyrimidine-4-carboxylate (18.0 g, 48.7 mmol) in MeOH (180 mL) and H₂O (90.0 mL) was added LiOH·H₂O (2.92 g, 121 mmol). The reaction was stirred at 20° C. for 2 hrs. The mixture was diluted with water (200 mL) and washed with MTBE (300 mL×2). The aqueous mixture was lyophilization to give the title compound (14.0 g, 41.3 mmol, 84.9% yield, 83.1% purity) as yellow solid.

Step C: N,6-dimethoxy-N-methyl-2-((S)-1-((S)-1-methylpyrrolidin-2-yl)ethoxy)pyrimidine-4-carboxamide. To a solution of lithium 6-methoxy-2-((S)-1-((S)-1-methylpyrrolidin-2-yl)ethoxy)pyrimidine-4-carboxylate (14.0 g, 49.7 mmol) and N,O-dimethylhydroxylamine hydrochloride (9.71 g, 99.5 mmol) in DMF (100 mL) were added DIEA (32.1 g, 248 mmol) and HATU (22.7 g, 59.7 mmol). The reaction was stirred at 25° C. for 1 hr. The mixture was diluted with water (500 mL) and extracted with dichloromethane (100 mL×2). The combined organic phases were washed with brine (500 mL), dried with anhydrous Na₂SO₄, filtered, concentrated, and purified with reversed-phase HPLC (0.1% FA condition) to the title compound (8.70 g, 26.8 mmol, 53.9% yield) as yellow oil.

Step D: (E)-N′-(3-cyano-4-(3-(6-methoxy-2-((S)-1-((S)-1-methylpyrrolidin-2-yl)ethoxy)pyrimidin-4-yl)-3-oxoprop-1-yn-1-yl)-4-methyl-4,5,6,7-tetrahydrobenzo[b]thiophen-2-yl)-N,N-dimethylformimidamide. To a solution of (E)-N′-(3-cyano-4-ethynyl-4-methyl-4,5,6,7-tetrahydrobenzo[b]thiophen-2-yl)-N,N-dimethylformimidamide (8.01 g, 29.50 mmol) in THF (50.0 mL) at −78° C. was added LiHMDS (1 M, 67.0 mL) dropwise. The mixture was stirred at −78° C. for 0.5 hr. A solution of N,6-dimethoxy-N-methyl-2-((S)-1-((S)-1-methylpyrrolidin-2-yl)ethoxy)pyrimidine-4-carboxamide (8.70 g, 26.8 mmol) in THF (50.0 mL) was added at −78° C. The reaction was stirred at −78° C. for 2 hrs. The residue was poured into NH₄Cl (aq) (50.0 mL) and stirred and the mixture was extracted with ethyl acetate (50.0 mL×2). The combined organic phases were washed with brine (50.0 mL), dried with anhydrous Na₂SO₄, filtered, and purified with column chromatography (SiO₂, Petroleum ether/Ethyl acetate: 10:1˜0:1) to give the title compound (11.0 g, 18.1 mmol, 67.5% yield, 84.2% purity) as yellow solid. LCMS: m/z=535.3 (M+1)⁺.

Step E: (E)-N′-(3-cyano-4-(3-(6-methoxy-2-((S)-1-((S)-1-methylpyrrolidin-2-yl)ethoxy)pyrimidin-4-yl) isothiazol-5-yl)-4-methyl-4,5,6,7-tetrahydrobenzo[b]thiophen-2-yl)-N,N-dimethylformimidamide. To a solution of (E)-N′-(3-cyano-4-(3-(6-methoxy-2-((S)-1-((S)-1-methylpyrrolidin-2-yl)ethoxy)pyrimidin-4-yl)-3-oxoprop-1-yn-1-yl)-4-methyl-4,5,6,7-tetrahydrobenzo[b]thiophen-2-yl)-N,N-dimethylformimidamide (1.00 g, 1.87 mmol) in MeOH (10.0 mL) was added NH₂OSO₃H (232 mg, 2.06 mmol) in portions. The reaction was stirred at 25° C. for 4 hrs. To the reaction mixture were added NaHS (262 mg, 4.68 mmol) and NaHCO₃ (172 mg, 2.06 mmol). The reaction was stirred at 50° C. for 1 hr. The residue was diluted with water (20.0 mL) and extracted with dichloromethane (20.0 mL×2). The combined organic phases were washed with brine (20.0 mL), dried with anhydrous Na₂SO₄, filtered, concentrated, and purified by reversed-phase HPLC (column: Phenomenex Luna C18 150*25 mm*10 um; mobile phase: [water (FA)-ACN]; B %: 23%-53%, 10 min) to give the title compound as yellow solid (200 mg, 0.33 mmol, 17.8% yield, 85.0% purity). ¹H NMR: (400 MHz MeOD-d⁴) δ 7.90 (s, 1H), 7.75 (d, J=10.8 Hz 1H), 7.17 (d, J=2.00 Hz 1H), 5.35-5.31 (m, 1H), 4.02 (s, 3H), 3.14 (s, 3H), 3.07 (s, 3H), 2.78-2.70 (m, 3H), 2.58-2.53 (m, 3H), 2.39-2.38 (m, 1H), 2.09-2.03 (m, 2H), 2.02-2.00 (m, 1H), 1.95 (s, 3H), 1.92-1.86 (m, 2H), 1.83-1.77 (m, 4H), 1.39-1.37 (m, 3H)

Step F: (E)-N′-(3-cyano-4-(3-(6-hydroxy-2-((S)-1-((S)-1-methylpyrrolidin-2-yl)ethoxy)pyrimidin-4-yl) isothiazol-5-yl)-4-methyl-4,5,6,7-tetrahydrobenzo[b]thiophen-2-yl)-N,N-dimethylformimidamide. To a mixture of (E)-N′-(3-cyano-4-(3-(6-methoxy-2-((S)-1-((S)-1-methylpyrrolidin-2-yl)ethoxy)pyrimidin-4-yl) isothiazol-5-yl)-4-methyl-4,5,6,7-tetrahydrobenzo[b]thiophen-2-yl)-N,N-dimethylformimidamide (2.00 g, 3.54 mmol,) and ACN (4.00 mL) were added TMSCl (1.92 g, 17.68 mmol, 2.24 mL) and NaI (2.65 g, 17.6 mmol) in one portion at 20° C. under N₂. The reaction was heated at 60° C. for 2 hrs. The reaction was poured into ice-water (20.0 mL) and the mixture was extracted with dichloromethane (10.0 mL×2). The combined organic phases were washed with brine (10.0 mL), dried with anhydrous Na₂SO₄, filtered, and concentrated, and purified by column chromatography to give the title compound (600 mg, 1.02 mmol, 28.9% yield, 94.7% purity) as yellow solid. LCMS: m/z=552.2 (M+1)⁺.

Step G: 6-(5-(3-cyano-2-(((E)-(dimethylamino)methylene)amino)-4-methyl-4,5,6,7-tetrahydrobenzo[b]thiophen-4-yl) isothiazol-3-yl)-2-((S)-1-((S)-1-methylpyrrolidin-2-yl)ethoxy)pyrimidin-4-yl trifluoromethanesulfonate. To a mixture of (E)-N′-(3-cyano-4-(3-(6-hydroxy-2-((S)-1-((S)-1-methylpyrrolidin-2-yl)ethoxy)pyrimidin-4-yl) isothiazol-5-yl)-4-methyl-4,5,6,7-tetrahydrobenzo[b]thiophen-2-yl)-N,N-dimethylformimidamide (400 mg, 725 μmol) and DCM (4.00 mL) were added DIEA (18.7 mg, 145 μmol) and Tf₂O (40.9 mg, 145 μmol, 23.92 μL) in one portion at 20° C. under N₂. The reaction was stirred for 4 hrs. The mixture was poured into ice-water (10.0 mL) and the aqueous mixture was extracted with dichloromethane (10.0 mL×2). The combined organic phases were washed with brine (10.0 mL), dried with anhydrous Na₂SO₄, filtered, and concentrated to give the title compound (400 mg, 584 μmol, 80.6% yield) as yellow solid.

Step H. (E)-N′-(3-cyano-4-(3-(6-((R)-3-hydroxy-3-methylpiperidin-1-yl)-2-((S)-1-((S)-1-methylpyrrolidin-2-yl)ethoxy)pyrimidin-4-yl) isothiazol-5-yl)-4-methyl-4,5,6,7-tetrahydrobenzo[b]thiophen-2-yl)-N,N-dimethylformimidamide. To a mixture of compound (R)-3-methylpiperidin-3-ol (25.9 mg, 171 μmol, HCl) and 6-(5-(3-cyano-2-(((E)-(dimethylamino)methylene)amino)-4-methyl-4,5,6,7-tetrahydrobenzo[b]thiophen-4-yl) isothiazol-3-yl)-2-((S)-1-((S)-1-methylpyrrolidin-2-yl)ethoxy)pyrimidin-4-yl trifluoromethanesulfonate (65.0 mg, 95.0 μmol) in THF (2.00 mL) was added DIEA (36.8 mg, 285 μmol, 49.6 μL) in one portion at 25° C. under N₂. The reaction was stirred at 25° C. for 2 hrs and poured into water (2.00 mL). The aqueous mixture was extracted with ethyl acetate (2.00 mL×3). The combined organic phase was washed with brine (2.00 mL), dried with anhydrous Na₂SO₄, filtered, and concentrated to give the title compound as yellow solid. LCMS: m/z=649.4 (M+1)⁺

Step I: 2-amino-4-(3-(6-((R)-3-hydroxy-3-methylpiperidin-1-yl)-2-((S)-1-((S)-1-methylpyrrolidin-2-yl)ethoxy)pyrimidin-4-yl) isothiazol-5-yl)-4-methyl-4,5,6,7-tetrahydrobenzo[b]thiophene-3-carbonitrile. To a solution of (E)-N′-(3-cyano-4-(3-(6-((R)-3-hydroxy-3-methylpiperidin-1-yl)-2-((S)-1-((S)-1-methylpyrrolidin-2-yl)ethoxy)pyrimidin-4-yl) isothiazol-5-yl)-4-methyl-4,5,6,7-tetrahydrobenzo[b]thiophen-2-yl)-N,N-dimethylformimidamide (50.0 mg, crude) in MeOH (2.00 mL) was added HCl (12 M, 1.00 mL) in one portion at 20° C. under N₂. The reaction was stirred at 100° C. for 2 hrs. The mixture was concentrated and purified with reversed-phase HPLC (column: Phenomenex Luna C18 150*25 mm*10 um; mobile phase: [water (FA)-ACN]; B %: 15%-45%, 9 min) to give the title compound as off-white solid (12.0 mg, 16.5 μmol, 17.3% yield, 98.8% purity, FA salt). LCMS: m/z=594.3 (M+1)⁺ ¹H NMR: 400 MHz MeOD-d⁴: δ7.73 (d, J=6.40 Hz, 1H), 7.17 (s, 1H), 5.30-5.27 (m, 1H), 3.83 (d, J=13.2 Hz, 1H), 3.47-3.41 (m, 2H), 3.25 (s, 1H), 3.02 (s, 1H), 2.69 (s, 3H), 2.62-2.59 (m, 3H), 2.05-2.02 (m, 4H), 1.91 (s, 3H), 1.88-1.75 (m, 7H), 1.65-1.62 (m, 2H), 1.40 (d, J=2.00 Hz, 1H), 1.26 (s, 3H).

### Example 8

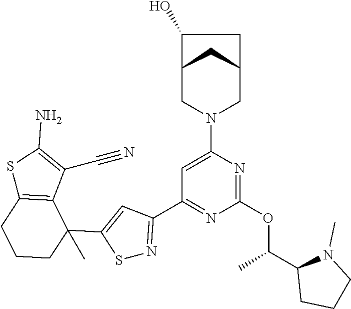 [SMILES: C[C@H](Oc1nc(-c2cc(C3(C)CCCc4sc(N)c(C#N)c43)sn2)cc(N2C[C@@H]3C[C@H](C2)[C@H](O)C3)n1)[C@@H]1CCCN1C]

### 2-amino-4-(3-(6-((1R,5R,6R)-6-hydroxy-3-azabicyclo[3.2.1]octan-3-yl)-2-((S)-1-((S)-1-methylpyrrolidin-2-yl)ethoxy)pyrimidin-4-yl) isothiazol-5-yl)-4-methyl-4,5,6,7-tetrahydrobenzo[b]thiophene-3-carbonitrile

Synthesized according to Example 7. The title compound was obtained as an off-white solid (formic acid salt). ¹H NMR: 400 MHZ MeOD-d⁴: δ7.73 (d, J=6.40 Hz, 1H), 7.12 (s, 1H), 5.32-5.28 (m, 1H), 4.32-4.29 (s, 1H), 3.48-3.36 (m, 2H), 3.17-3.08 (m, 3H), 2.77 (s, 3H), 2.74-2.62 (m, 1H), 2.61-2.58 (m, 2H), 2.36 (s, 1H), 2.28-2.19 (m, 3H), 2.05-2.03 (m, 2H), 1.99-1.95 (m, 2H), 1.90 (s, 3H), 1.87-1.76 (m, 5H), 1.42 (d, J=6.00 Hz, 3H), 1.28-1.24 (m, 2H). LCMS: m/z=606.3 (M+1)⁺.

### Example 9

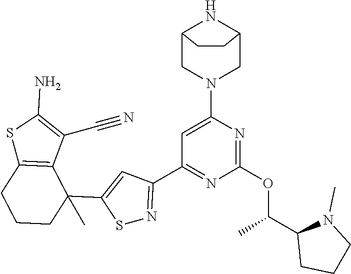 [SMILES: C[C@H](Oc1nc(-c2cc(C3(C)CCCc4sc(N)c(C#N)c43)sn2)cc(N2CC3CCC(C2)N3)n1)[C@@H]1CCCN1C]

### 4-(3-(6-((1R,5S)-3,8-diazabicyclo[3.2.1]octan-3-yl)-2-((S)-1-((S)-1-methylpyrrolidin-2-yl)ethoxy)pyrimidin-4-yl) isothiazol-5-yl)-2-amino-4-methyl-4,5,6,7-tetrahydrobenzo[b]thiophene-3-carbonitrile

Synthesized according to Example 7. The title compound was obtained as an off-white solid (formic acid salt). ¹H NMR: 400 MHZ MeOD-d⁴: δ 8.55 (s, 1H), 7.75 (d, J=7.20 Hz, 1H), 7.15 (s, 1H), 5.30-5.27 (m, 1H), 3.79 (s, 2H), 3.41-3.39 (m, 3H), 3.25-3.24 (m, 2H), 2.85 (s, 4H), 2.61 (t, J=6.40 Hz 2H), 2.26 (s, 1H), 2.06-2.04 (m, 3H), 1.98-1.93 (m, 3H), 1.91-1.88 (m, 5H), 1.82-1.80 (m, 4H), 1.44 (d, J=6.00 Hz, 3H). LCMS: m/z=591.3 (M+1)⁺.

### Example 10

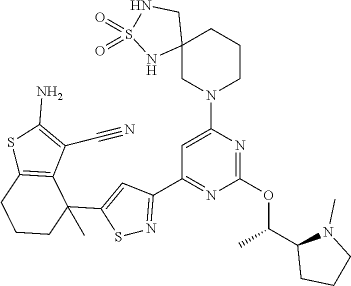 [SMILES: C[C@H](Oc1nc(-c2cc(C3(C)CCCc4sc(N)c(C#N)c43)sn2)cc(N2CCCC3(CNS(=O)(=O)N3)C2)n1)[C@@H]1CCCN1C]

### 2-amino-4-(3-(6-(2,2-dioxido-2-thia-1,3,7-triazaspiro[4.5]decan-7-yl)-2-((S)-1-((S)-1-methylpyrrolidin-2-yl)ethoxy)pyrimidin-4-yl) isothiazol-5-yl)-4-methyl-4,5,6,7-tetrahydrobenzo[b]thiophene-3-carbonitrile

Synthesized according to Example 7. The title compound was obtained as an off-white solid (formic acid salt). ¹H NMR: 400 MHZ MeOD-d⁴: δ7.76-7.74 (m, 1H), 7.23-7.22 (m, 1H), 5.37-5.34 (m, 1H), 3.72 (s, 1H), 3.39-3.35 (m, 4H), 3.19-3.16 (m, 2H), 2.74-2.61 (m, 4H), 2.61 (s, 2H), 2.31-2.15 (m, 1H), 2.03-1.96 (m, 6H), 1.91 (s, 3H), 1.86-1.81 (m, 6H), 1.44-1.39 (m, 3H). LCMS: m/z=670.2 (M+1)⁺.

### Example 11

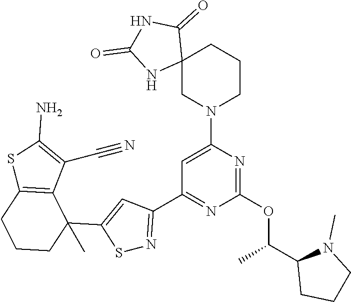 [SMILES: C[C@H](Oc1nc(-c2cc(C3(C)CCCc4sc(N)c(C#N)c43)sn2)cc(N2CCCC3(C2)NC(=O)NC3=O)n1)[C@@H]1CCCN1C]

### 2-amino-4-(3-(6-(2,4-dioxo-1,3,7-triazaspiro[4.5]decan-7-yl)-2-((S)-1-((S)-1-methylpyrrolidin-2-yl)ethoxy)pyrimidin-4-yl) isothiazol-5-yl)-4-methyl-4,5,6,7-tetrahydrobenzo[b]thiophene-3-carbonitrile

Synthesized according to Example 7. The title compound was obtained as an off-white solid (formic acid salt). ¹H NMR: 400 MHZ MeOD-d⁴: δ7.76-7.74 (m, 1H), 7.24 (s, 1H), 5.25-5.22 (m, 1H), 3.55-3.46 (m, 2H), 2.92-2.88 (m, 1H), 2.82 (s, 3H), 2.63-2.59 (m, 3H), 2.23-2.17 (m, 3H), 2.05-1.98 (m, 6H), 1.91 (s, 3H), 1.87-1.80 (m, 4H), 1.42 (d, J=6.00 Hz, 3H), 1.35-1.30 (m, 2H). LCMS: m/z=648.3 (M+1)⁺.

### Example 12

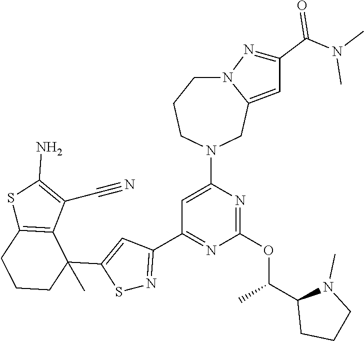 [SMILES: C[C@H](Oc1nc(-c2cc(C3(C)CCCc4sc(N)c(C#N)c43)sn2)cc(N2CCCn3nc(C(=O)N(C)C)cc3C2)n1)[C@@H]1CCCN1C]

### 5-(6-(5-(2-amino-3-cyano-4-methyl-4,5,6,7-tetrahydrobenzo[b]thiophen-4-yl) isothiazol-3-yl)-2-((S)-1-((S)-1-methylpyrrolidin-2-yl)ethoxy)pyrimidin-4-yl)-N,N-dimethyl-5,6,7,8-tetrahydro-4H-pyrazolo[1,5-a][1,4]diazepine-2-carboxamide

Synthesized according to Example 7. The title compound was obtained as an off-white solid (formic acid salt). ¹H NMR: 400 MHz MeOD-d⁴: δ 7.62 (d, J=6.40 Hz, 1H), 7.13 (s, 1H), 6.61 (s, 1H), 5.18 (s, 1H), 4.05 (s, 2H), 3.34-3.22 (m, 2H), 3.17 (s, 3H), 2.95 (s, 3H), 2.74 (s, 4H), 2.50-2.41 (m, 3H), 2.05-2.02 (m, 2H), 1.94-1.90 (m, 7H), 1.78 (s, 3H), 1.71-1.66 (m, 2H), 1.34-1.33 (m, 3H), 1.31-1.19 (m, 2H). LCMS: m/z=687.3 (M+1)⁻.

### Example 13

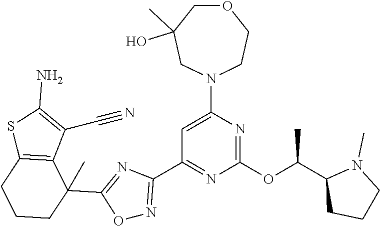 [SMILES: C[C@H](Oc1nc(-c2noc(C3(C)CCCc4sc(N)c(C#N)c43)n2)cc(N2CCOCC(C)(O)C2)n1)[C@@H]1CCCN1C]

### 2-amino-4-(3-(6-(6-hydroxy-6-methyl-1,4-oxazepan-4-yl)-2-((S)-1-((S)-1-methylpyrrolidin-2-yl)ethoxy)pyrimidin-4-yl)-1,2,4-oxadiazol-5-yl)-4-methyl-4,5,6,7-tetrahydrobenzo[b]thiophene-3-carbonitrile

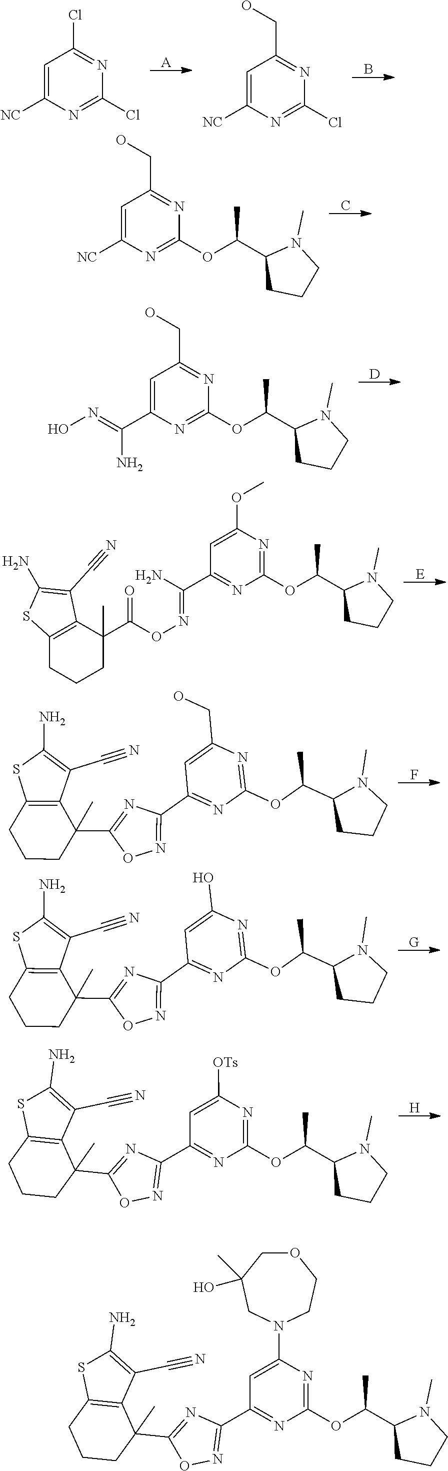

Step A. 2-chloro-6-methoxypyrimidine-4-carbonitrile: To a solution of 2,6-dichloropyrimidine-4-carbonitrile (18.6 g, 1.0 equiv) in MeOH (200 mL) was added CH₃ONa (19.3 g, 30% purity, 1.0 equiv). The reaction was stirred at −40° C. for 2 hours. The mixture was diluted with water (500 mL) and extracted with ethyl acetate (2× 500 mL). The combined organic layers were washed with brine (500 mL), dried over anhydrous sodium sulfate, concentrated, and purified with column chromatography [SiO₂, petroleum ether/ethyl acetate=20/1 to 5/1] to afford the title compound (15.8 g, 87% yield) as yellow solid; ¹H NMR (400 MHZ, CHLOROFORM-d) δ=7.04 (s, 1H), 4.10 (s, 3H).

Step B. 6-methoxy-2-((S)-1-((S)-1-methylpyrrolidin-2-yl)ethoxy)pyrimidine-4-carbonitrile: To a solution of 2-chloro-6-methoxypyrimidine-4-carbonitrile (15.8 g, 1.0 equiv) and (1S)-1-[(2S)-1-methylpyrrolidin-2-yl]ethanol (12.0 g, 1.0 equiv) in acetonitrile (200 mL) was added DIEA (36.1 g, 3.0 equiv). The reaction was stirred at 70° C. for 12 hours. The mixture was diluted with water (500 mL) and extracted with ethyl acetate (2× 500 mL). The combined organic layers were washed with brine (500 mL), dried over anhydrous sodium sulfate, concentrated, and purified with column chromatography [SiO₂, petroleum ether/ethyl acetate=20/1 to 0/1] to afford the title compound (17.8 g, 73% yield) as yellow oil; LCMS (ESI, M+1): m/z=263.3.

Step C. (Z)—N′-hydroxy-6-methoxy-2-((S)-1-((S)-1-methylpyrrolidin-2-yl)ethoxy)pyrimidine-4-carboximidamide: To a solution of 6-methoxy-2-((S)-1-((S)-1-methylpyrrolidin-2-yl)ethoxy)pyrimidine-4-carbonitrile (12.5 g, 1.0 equiv) in ethanol (300 mL) were added NH₂OH·HCl (4.30 g, 1.3 equiv) and Na₂CO₃ (13.1 g, 2.6 equiv) at 0° C. The reaction was stirred at 25° C. for 3 hours. The mixture was concentrated, diluted with water (50 mL), and extracted with ethyl acetate (2× 50 mL). The combined organic layers were washed with brine (50 mL), dried over anhydrous sodium sulfate, and concentrated to afford the title compound (13.8 g, crude) as yellow solid; LCMS (ESI, M+1); m/z=296.2.

Step D. (Z)—N′-((2-amino-3-cyano-4-methyl-4,5,6,7-tetrahydrobenzo[b]thiophene-4-carbonyl)oxy)-6-methoxy-2-((S)-1-((S)-1-methylpyrrolidin-2-yl)ethoxy)pyrimidine-4-carboximidamide: To a solution of 2-amino-3-cyano-4-methyl-6,7-dihydro-5H-benzothiophene-4-carboxylic acid (9.94 g, 0.9 equiv) in DMF (150 mL) were added TEA (14.2 g, 3.0 equiv), HOBt (9.47 g, 1.5 equiv) and EDCI (11.2 g, 1.3 equiv). The reaction was stirred at 25° C. for 0.5 hours. Then a solution of (Z)—N′-hydroxy-6-methoxy-2-((S)-1-((S)-1-methylpyrrolidin-2-yl)ethoxy)pyrimidine-4-carboximidamide (13.8 g, 1.0 equiv) in DMF (150 mL) was added to the mixture. The reaction was stirred at 45° C. for 12 hours. The mixture was diluted with water (1 L) and extracted with ethyl acetate (2×1 L). The combined organic layers were washed with brine (1 L), dried over anhydrous sodium sulfate, concentrated, and purified with column chromatography [SiO₂, petroleum ether/ethyl acetate=10/1 to 0/1] to afford the title compound (20.0 g, 71% yield) as yellow solid; LCMS (ESI, M+1): m/z=514.2.

Step E. 2-amino-4-(3-(6-methoxy-2-((S)-1-((S)-1-methylpyrrolidin-2-yl)ethoxy)pyrimidin-4-yl)-1,2,4-oxadiazol-5-yl)-4-methyl-4,5,6,7-tetrahydrobenzo[b]thiophene-3-carbonitrile: To a solution of (Z)—N′-((2-amino-3-cyano-4-methyl-4,5,6,7-tetrahydrobenzo[b]thiophene-4-carbonyl)oxy)-6-methoxy-2-((S)-1-((S)-1-methylpyrrolidin-2-yl)ethoxy)pyrimidine-4-carboximidamide (20.0 g, 1.0 equiv) in THF (200 mL) was added Cs₂CO₃ (21.6 g, 2.0 equiv). The reaction was stirred at 70° C. for 1 hour. The mixture was diluted with water (200 mL) and extracted with ethyl acetate (2×200 mL). The combined organic layers were washed with brine (200 mL), dried over anhydrous sodium sulfate, concentrated, and purified with column chromatography [SiO₂, petroleum ether/ethyl acetate=10/1 to 0/1] to afford the title compound (15.0 g, 74% yield) as yellow solid; LCMS (ESI, M+1): m/z=496.4.

Step F. 2-amino-4-(3-(6-hydroxy-2-((S)-1-((S)-1-methylpyrrolidin-2-yl)ethoxy)pyrimidin-4-yl)-1,2,4-oxadiazol-5-yl)-4-methyl-4,5,6,7-tetrahydrobenzo[b]thiophene-3-carbonitrile: To a solution of 2-amino-4-(3-(6-methoxy-2-((S)-1-((S)-1-methylpyrrolidin-2-yl)ethoxy)pyrimidin-4-yl)-1,2,4-oxadiazol-5-yl)-4-methyl-4,5,6,7-tetrahydrobenzo[b]thiophene-3-carbonitrile (8.00 g, 1.0 equiv) in DMAc (80 mL) was added NaSEt (5.47 g, 5.0 equiv). The reaction was stirred at 60° C. for 1 hour. The mixture was diluted with water (250 mL) and washed with ethyl acetate (2×250 mL). The aqueous phase was concentrated and purified with prep-HPLC [column: Phenomenex luna C18 (250×70 mm, 10 μm); mobile phase: water (FA)-ACN; B %: 7%-37% 20 min] to afford the title compound (3.80 g, 61% yield) as yellow solid; LCMS (ESI, M+1): m/z=482.3.

Step G. 6-(5-(2-amino-3-cyano-4-methyl-4,5,6,7-tetrahydrobenzo[b]thiophen-4-yl)-1,2,4-oxadiazol-3-yl)-2-((S)-1-((S)-1-methylpyrrolidin-2-yl)ethoxy)pyrimidin-4-yl 4-methylbenzenesulfonate: To a solution of 2-amino-4-(3-(6-hydroxy-2-((S)-1-((S)-1-methylpyrrolidin-2-yl)ethoxy)pyrimidin-4-yl)-1,2,4-oxadiazol-5-yl)-4-methyl-4,5,6,7-tetrahydrobenzo[b]thiophene-3-carbonitrile (3.80 g, 1.0 equiv) and TEA (2.40 g, 3.0 equiv) in DCM (40 mL) was added 4-methylbenzenesulfonyl chloride (2.26 g, 1.5 equiv) dropwise at 0° C. The reaction was stirred at 20° C. for 1 hour. The mixture was diluted with water (200 mL) and extracted with DCM (2×200 mL). The combined organic layers were washed with brine (200 mL), dried over anhydrous sodium sulfate, concentrated, and purified with column chromatography [Al₂O₃, ethyl acetate] to afford the title compound (4.00 g, 80% yield) as yellow solid; LCMS (ESI, M+1): m/z=636.2.

Step H. 2-amino-4-(3-(6-(6-hydroxy-6-methyl-1,4-oxazepan-4-yl)-2-((S)-1-((S)-1-methylpyrrolidin-2-yl)ethoxy)pyrimidin-4-yl)-1,2,4-oxadiazol-5-yl)-4-methyl-4,5,6,7-tetrahydrobenzo[b]thiophene-3-carbonitrile: To a solution of 6-(5-(2-amino-3-cyano-4-methyl-4,5,6,7-tetrahydrobenzo[b]thiophen-4-yl)-1,2,4-oxadiazol-3-yl)-2-((S)-1-((S)-1-methylpyrrolidin-2-yl)ethoxy)pyrimidin-4-yl 4-methylbenzenesulfonate (100 mg, 1.0 equiv) and 4 Å molecular sieve (100 mg) in DMF (1 mL) were added 6-methyl-1,4-oxazepan-6-ol (39.5, 1.5 equiv, HCl) and DIEA (102 mg, 5.0 equiv). The reaction was stirred at 80° C. for 1 hour. The mixture was filtered. The filtrate was diluted with water (5 mL) and extracted with ethyl acetate (3×8 mL). The combined organic layers were washed with brine (10 mL), dried over anhydrous sodium sulfate, concentrated and purified with prep-HPLC [column: Waters Xbridge 150×25 mm×5 um; mobile phase: water (NH₄HCO₃)-ACN; gradient: 30%-60% B over 9 min] to afford the title compound (5.50 mg, 5.8% yield) as off-white solid; ¹H NMR (400 MHZ, METHANOL-d₄) δ=7.36-7.04 (m, 1H), 5.29-5.19 (m, 1H), 4.05-3.82 (m, 4H), 3.75-3.50 (m, 4H), 3.10-3.05 (m, 1H), 2.75-2.67 (m, 1H), 2.63 (br t, J=6.0 Hz, 2H), 2.54 (s, 3H), 2.37 (q, J=8.4 Hz, 1H), 2.28-2.20 (m, 1H), 2.05-1.95 (m, 4H), 1.90 (s, 3H), 1.83-1.74 (m, 3H), 1.37-1.33 (m, 3H), 1.26 (s, 3H); LCMS (ESI, M+1): m/z=595.4.

### Example A

### KRas Binding Assay

This Example illustrates that exemplary compounds of the present invention bind to KRas and are capable of displacing a labeled tracer ligand occupying the KRas binding site. KRas^{WT}, KRas^{G12A}, KRas^{G12C}, KRas^{G12D}, KRas^{G12R}, KRas^{G12S}, KRas^{G12V}, KRas^{G13D}, or KRas^{Q61H} was used in the assay.

The ability of a compound to bind to KRas was measured using a TR-FRET displacement assay. Biotinylated KRas (corresponding to amino acids 1-169, produced at Accelegan Inc.) was incubated with custom made Cy5 labelled tracer, terbium streptavidin (Cisbio Inc.) and compound (1% DMSO final) in buffer (50 mM HEPES, pH 7.5, 5 mM MgCl₂, 0.005% Tween-20 and 1 mM DTT). After a 60-minute incubation at room temperature, the reaction was measured using a BMG LABTECH CLARIO star Plus via TR-FRET. 100 percent of control (POC) is determined by using a DMSO control and 0 POC is determined using a concentration of control compound that completely inhibits binding of the tracer to KRas. The POC values were fit to a 4-parameter IC₅₀ equation and the IC₅₀ value reported.

TABLE 1
Binding to KRas (IC₅₀ nM) by Exemplary Compounds of Formula (I)

| Ex. | | | | | | | | | |
|---|---|---|---|---|---|---|---|---|---|
| No. | G12D | G12V | G12R | WT | G13D | Q61H | G12A | G12C | G12S |
| 1 | 42 | 43 | 28 | 22 | 74 | 117 | 82 | 106 | 90 |
| 2 | 18 | 6 | 4 | 14 | 37 | 36 | 49 | 54 | 41 |
| 3 | 18 | 767 | 728 | 195 | 315 | 656 | 879 | 1057 | 912 |
| 4 | 6 | 5 | 4 | 10 | 24 | 26 | 29 | 26 | 23 |
| 5 | 413 | 62 | 38 | 69 | 269 | 233 | 250 | 313 | 265 |
| 6 | 1835 | 746 | 669 | 474 | 1078 | 1110 | 1587 | 1107 | 1516 |
| 7 | 127 | 46 | 23 | 129 | 128 | 170 | 132 | 174 | 158 |
| 8 | 505 | 96 | 59 | 160 | 167 | 224 | 204 | 275 | 296 |
| 9 | 36 | 1042 | 1340 | 566 | 1322 | 2285 | 3076 | 3493 | 3423 |
| 10 | 3 | 4 | <2 | 31 | 24 | 9 | 35 | 15 | 25 |
| 11 | 93 | 76 | 34 | 326 | 190 | 263 | 240 | 262 | 188 |
| 12 | 623 | 163 | 156 | 417 | 584 | 533 | 551 | 457 | 596 |
| 13 | 11 | 6 | 4 | | | | | | |

### Example B

### Inhibition of KRas Phosphorylation of ERK (HTRF) by Exemplary Compounds of Formula (I)

Cisbio HTRF Advanced pERK Assay Catalog #64AERPEH
Cells: MKN1, PSN1

### Procedure:

Day 1: Seed 6,000 cells/well-25 μl/well in 384-well white solid bottom plate; RPM1_10% FBS. Incubate overnight at 37° C./5% CO2.
Day 2: Echo transfer 25 nl of 10 mM compound 10 point dilution at 1:3 (Cf=10 uM) and incubate for 3 hour at 37° C./5% CO2.
Add 8.5 μl/well of 4× Lysis Buffer/25× Blocking reagent (do not dump media) and incubate for 30 min at room temperature on shaker.
Add conjugate mixture of 4.25 μl/well 1×-pERK-D2 and 1×-pERK-K diluted in Detection Buffer for a total of 8.5 μl/well.
Incubate for 4 hours at room temperature covered.
Read HTRF using ClarioStar
   Cells: ASPC1, H727, A549, H460, HCT116, H358
      Culture/Assay media: RPMI-1640+10% FBS

### Procedure:

### Cell Seeding

1. To harvest cells from flask using 0.05% Trypsin/EDTA solution. Add 10 mL of media to stop trypsinizing. Pipette the cells into a conical bottom 50 mL centrifuge tube and centrifuge 5 min×1000 rpm.
2. Re-suspend the cell pellet in media, take a cell count, and then adjust the cell density using fresh media.
3. Seed 6,000 cells into cell culture plate with 50 μL media. The
4. Incubate cell plate overnight in a 37° C., 5% CO2 incubator.

### Compound Titrations

1. Use Tecan to complete the compound addition. Compounds start from 10 uM top, 3-fold dilution, and 10 doses. The final DMSO concentration is 0.8%. Dispensed 0.2 uM Trametinib as Min control.
2. Incubate cell plate for 3 hrs in the incubator.
   Detection with Cisbio pERK HTRF Kit
1. Dilute 1 volume of 4× lysis buffer with 3 volumes of deionized water. Then, add 100× the blocking reagent. Keep lysis buffer on the ice.
2. At the end of the compound treatment, flick-off the media.
3. Add 35 μL of lysis buffer per well using a Multidrop Combi. Then place on a plate agitator shaking at 300 rpm at 4° C. for 40 mins.
4. Make up the HTRF antibody buffer. For each assay plate, mix 50 μL of d2-conjugate antibody with 950 μL of detection buffer. Similarly, mix 50 μL of Cryptate antibody with 950 μL of detection buffer. Then mix the two diluted antibodies together.
5. Dispense 3.4 μL the antibody buffer to wells of an empty assay plate. Seal the plate and centrifuge plate 30 sec×1000 rpm.
6. At the end of the 4° C. lysis, centrifuge the lysate plates 3 mins×1500 rpm.
7. Use the Bravo to transfer 13.6 μL of lysate from cell culture plate to assay plate. Then incubate assay plate for 2 hrs at room temperature.
8. At the end of incubation, read plate on the Envision after centrifuging plate 30 sec×1000 rpm.

### Table 2

### Inhibition (HTRF IC₅₀ nM) of KRas-mediated Phosphorylation of ERK by Exemplary Compounds of Formula (I)

| | | | | | | | | | |
|---|---|---|---|---|---|---|---|---|---|
| Ex. | | | | | | | | | |
| No. | AsPC-1 | H727 | MKN1 | HCT116 | A549 | H460 | H358 | H2009 | PSN1 |

| | | | | | | | | |
|---|---|---|---|---|---|---|---|---|
| 1 | 3738 | 6445 | 593 | | | | | >10000 |
| 3 | 5945 | >10000 | >10000 | | | | | >10000 |
| 6 | 7724 | >10000 | | | | | | |
| 7 | 5835 | 4474 | | | | | | |
| 8 | 7401 | 3376 | | | | | | |
| 9 | 3011 | 10000 | | | | | | |
| 10 | 898 | 3895 | 10 | 962 | 727 | 288 | 216 | 9303 |
| 13 | 507 | 469 | 44 | 899 | 689 | 394 | 43 | >10000 |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A compound of Formula (I): 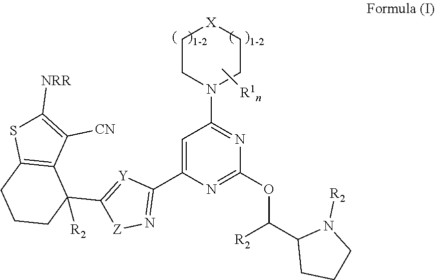
or a pharmaceutically acceptable salt thereof, wherein:
X is CR, O or N;
Y is CR or N;
Z is O or S;
n is an integer from 1 to 4;
each R is independently H or C1-C3 alkyl;
R1 is C1-C3 alkyl or hydroxy; or
n is at least two, and two R¹s optionally join to form a methylene or ethylene bridge; or
n is at least 2, and two R¹s optionally join to form a spiro or fused ring, where the ring is heterocyclic or heteroaryl, and where the ring is optionally substituted with 1-2 substituents selected from oxo and —C(O)N(CH3)(CH3); and
each R² is independently C1-C3 alkyl.

2. The compound or salt of claim 1, wherein n is 2, and two R¹s form a saturated heterocyclic ring containing S and N atoms.

3. The compound or salt of claim 2, wherein the saturated heterocyclic ring formed by the two R¹s is substituted with two oxos.

4. The compound or salt of claim 1, wherein n is 2, one R¹ is OH and the other R¹ is CH₃.

5. A compound selected from: 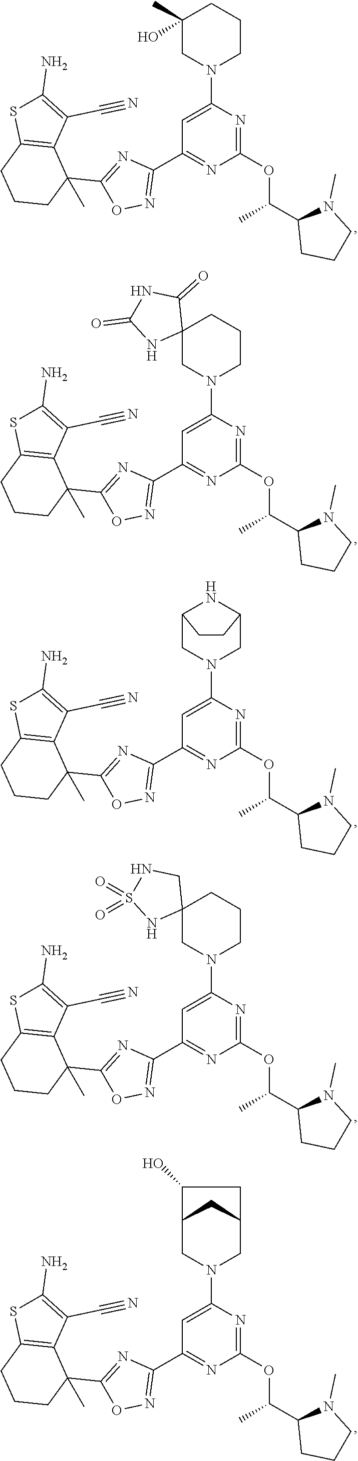 [SMILES: C[C@H](Oc1nc(-c2noc(C3(C)CCCc4sc(N)c(C#N)c43)n2)cc(N2CCC[C@@](C)(O)C2)n1)[C@@H]1CCCN1C ; C[C@H](Oc1nc(-c2noc(C3(C)CCCc4sc(N)c(C#N)c43)n2)cc(N2CCCC3(C2)NC(=O)NC3=O)n1)[C@@H]1CCCN1C ; C[C@H](Oc1nc(-c2noc(C3(C)CCCc4sc(N)c(C#N)c43)n2)cc(N2CC3CCC(C2)N3)n1)[C@@H]1CCCN1C ; C[C@H](Oc1nc(-c2noc(C3(C)CCCc4sc(N)c(C#N)c43)n2)cc(N2CCCC3(CNS(=O)(=O)N3)C2)n1)[C@@H]1CCCN1C ; C[C@H](Oc1nc(-c2noc(C3(C)CCCc4sc(N)c(C#N)c43)n2)cc(N2C[C@@H]3C[C@H](C2)[C@H](O)C3)n1)[C@@H]1CCCN1C] 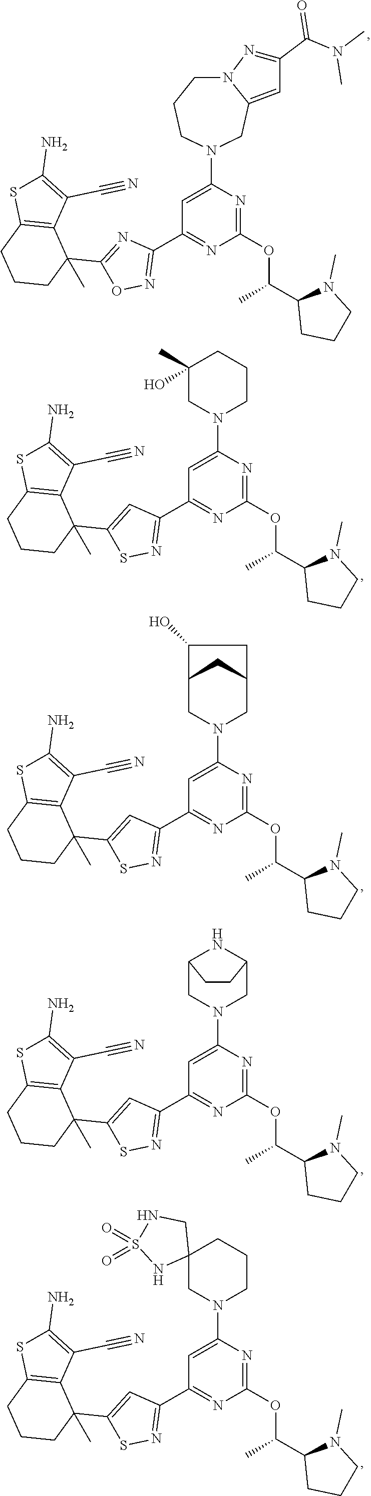 [SMILES: C[C@H](Oc1nc(-c2noc(C3(C)CCCc4sc(N)c(C#N)c43)n2)cc(N2CCCn3nc(C(=O)N(C)C)cc3C2)n1)[C@@H]1CCCN1C ; C[C@H](Oc1nc(-c2cc(C3(C)CCCc4sc(N)c(C#N)c43)sn2)cc(N2CCC[C@@](C)(O)C2)n1)[C@@H]1CCCN1C ; C[C@H](Oc1nc(-c2cc(C3(C)CCCc4sc(N)c(C#N)c43)sn2)cc(N2C[C@@H]3C[C@H](C2)[C@H](O)C3)n1)[C@@H]1CCCN1C ; C[C@H](Oc1nc(-c2cc(C3(C)CCCc4sc(N)c(C#N)c43)sn2)cc(N2CC3CCC(C2)N3)n1)[C@@H]1CCCN1C ; C[C@H](Oc1nc(-c2cc(C3(C)CCCc4sc(N)c(C#N)c43)sn2)cc(N2CCCC3(CNS(=O)(=O)N3)C2)n1)[C@@H]1CCCN1C] 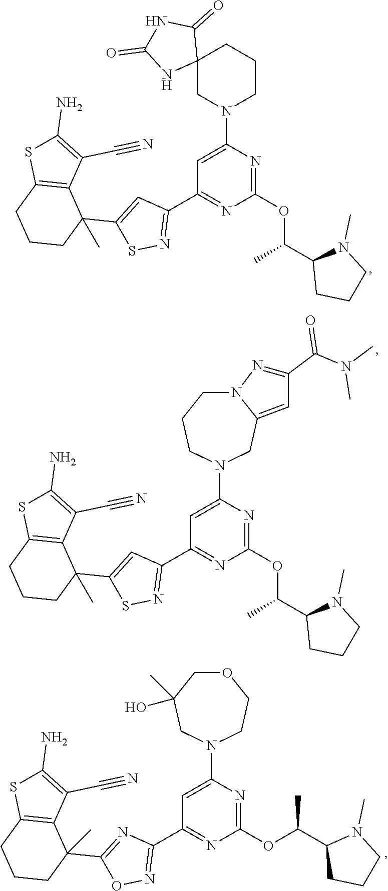
and pharmaceutically acceptable salts thereof.

6. A pharmaceutical composition, comprising a therapeutically effective amount of a compound of any one of claims 1-5 or a pharmaceutically acceptable salt thereof, and a pharmaceutically acceptable excipient.

7. A method for inhibiting the wild type KRas, KRas G12A, KRas G12C, KRas G12D, KRas G12R, KRas G12S, KRas G12V, KRas G13D or KRas Q61H activity in a cell, comprising contacting the cell in which inhibition of KRas activity is desired with an effective amount of a compound of according to any one of claims 1-5 or a pharmaceutically acceptable salt thereof, or the pharmaceutical composition of claim 6.

8. A method for treating cancer comprising administering to a patient having cancer a therapeutically effective amount of a compound according to any one of claims 1-5 or a pharmaceutically acceptable salt thereof, or the pharmaceutical composition of claim 6.

9. The method of claim 8, wherein the therapeutically effective amount of the compound is between about 0.01 to 100 mg/kg per day.

10. The method of claim 9, wherein the therapeutically effective amount of the compound is between about 0.1 to 50 mg/kg per day.

11. The method of claim 8, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial ‘carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

12. The method of claim 11, wherein the cancer is a KRas G12A-associated cancer.

13. The method of claim 11, wherein the cancer is a KRas G12C-associated cancer.

14. The method of claim 11, wherein the cancer is a KRas G12D-associated cancer.

15. The method of claim 11, wherein the cancer is a KRas G12R-associated cancer.

16. The method of claim 11, wherein the cancer is a KRas G12S-associated cancer.

17. The method of claim 11, wherein the cancer is a KRas G12V-associated cancer.

18. The method of claim 11, wherein the cancer is a KRas G13D-associated cancer.

19. The method of claim 11, wherein the cancer is a KRas Q61H-associated cancer.

20. The method of claim 11, wherein the cancer is a KRas G12A-associated cancer.

21. The method of claim 11, wherein the cancer is associated with at least one of wild type KRas, KRas G12A, KRas G12C, KRas G12D, KRas G12R, KRas G12S, KRas G12V, KRas G13D or KRas Q61H.

22. The method of any of claims 7-21, wherein the cancer is non-small cell lung cancer, small cell lung cancer, colorectal cancer, rectal cancer or pancreatic cancer.

23. A method for treating cancer in a patient in need thereof, the method comprising (a) determining that the cancer is associated with wild type KRas or a KRas G12A, KRas G12C, KRas G12D, KRas G12R, KRas G12S, KRas G12V, KRas G13D or KRas Q61H mutation; and (b) administering to the patient a therapeutically effective amount of a compound according to any one of claims 1-5 or a pharmaceutically acceptable salt thereof, or the pharmaceutical composition of claim 6.

24. The method of any one of claims 7-23, wherein the administering is done via a route selected from the group consisting of parenteral, intraperitoneal, intradermal, intracardiac, intraventricular, intracranial, intracerebrospinal, intrasynovial, intrathecal administration, intramuscular injection, intravitreous injection, intravenous injection, intra-arterial injection, oral, buccal, sublingual, transdermal, topical, intratracheal, intrarectal, subcutaneous, and topical administration.

25. The method of claim 24, wherein the administration route is oral.

26. The method of claim 24, wherein the administration is intravenous injection.

27. The method of claim 24, wherein the administration route is intramuscular injection.

28. The method of claim 24, wherein the administration route utilizes a delivery device.

29. The method of claim 24, wherein administration is done in a hospital setting.
